(19) United States
(12) Patent Application Publication
(10) Pub. No.: US 2021/0177341 A1
(43) Pub. Date: Jun. 17, 2021

(54) UTILIZING WEARABLE ELECTRONIC DEVICES AT A WORKSITE

(71) Applicant: SCHLUMBERGER TECHNOLOGY CORPORATION, Sugar Land, TX (US)

(72) Inventors: Manat Singh, Houston, TX (US); Vishwanathan Parmeshwar, Houston, TX (US)

(21) Appl. No.: 16/761,343

(22) PCT Filed: Nov. 2, 2018
(86) PCT No.: PCT/US2018/058867

§ 371 Date: May 4, 2020

Related U.S. Application Data
(60) Provisional application No. 62/581,065, filed on Nov. 3, 2017.

(51) Int. Cl. A61B 5/00; A61B 5/11; A61B 5/16

(52) U.S. Cl. CPC A61B 5/4809; A61B 5/1118; A61B 5/0002; A61B 5/1123; A61B 2503/20; A61B 5/6805; A61B 5/168; A61B 5/7405; A61B 5/7455; A61B 5/6803

(57) ABSTRACT

Apparatus and methods for utilizing wearable electronic devices at a worksite. A method may include donning an electronic device that contains or is in wireless communication with a processing device that includes a processor and a memory storing computer program code, then performing an action or having an experience, wherein the performed action or experience is detected by the donned electronic device, and then perceiving a sensory signal output by the donned electronic device, wherein the sensory signal output is caused by the processing device based on the detected action or experience.

### CROSS-REFERENCE TO RELATED APPLICATIONS

This application claims priority to and the benefit of U.S. Provisional Application No. 62/581,065, titled “WORK OPTIMIZATION AND SAFETY USING WEARABLE TECHNOLOGY,” filed Nov. 3, 2017, the entire disclosure of which is hereby incorporated herein by reference.

### BACKGROUND OF THE DISCLOSURE

Wells are generally drilled into the ground or ocean bed to recover natural deposits of oil, gas, and other materials that are trapped in subterranean formations. Well construction operations (e.g., drilling operations) may be performed at a wellsite by a drilling system having various automated surface and subterranean equipment operating in a coordinated manner. For example, a drive mechanism, such as a top drive or rotary table located at a wellsite surface, can be utilized to rotate and advance a drill string into a subterranean formation to drill a wellbore. The drill string may include a plurality of drill pipes coupled together and terminating with a drill bit. Length of the drill string may be increased by adding additional drill pipes while depth of the wellbore increases. Drilling fluid may be pumped from the wellsite surface down through the drill string to the drill bit. The drilling fluid lubricates and cools the drill bit, and carries drill cuttings from the wellbore back to the wellsite surface. The drilling fluid returning to the surface may then be cleaned and again pumped through the drill string. The equipment of the drilling system may be grouped into various subsystems, wherein each subsystem performs a different operation controlled by a corresponding local and/or a remotely located controller.

During drilling operations, the automated equipment of the drilling system is continuously being worked on by human wellsite workers (e.g., drillers, roughnecks, maintenance crew, etc.). Such work includes operating the equipment to perform the well construction operations and conducting maintenance activities at the wellsite or in a workshop to preserve condition (i.e., health) of the equipment. Although the automated equipment increases efficiency of the well construction operations, such equipment poses a safety hazard to the wellsite workers. For example, serious injuries may be sustained by a wellsite worker who, while working alongside an automated machine, is struck or pushed by an automated machine executing an automated sequence during well construction operations.

Maintenance activities may be foreseeable and periodic or unpredictable and in response to an untimely failure. Periodic maintenance schedules can vary considerably from daily maintenance of equipment, such as greasing, to monthly changing out of consumables, such as filters, to complete overhauling of the equipment after a predetermined period of use. Each of these activities can take anywhere from minutes to days to complete. Furthermore, drilling rigs or remote maintenance centers do not have the capability to accurately monitor and/or plan maintenance activities, resulting in variability, non-standardization, and inefficiency of maintenance activities, particularly in terms of time taken to accomplish such maintenance activities.

### SUMMARY OF THE DISCLOSURE

This summary is provided to introduce a selection of concepts that are further described below in the detailed description. This summary is not intended to identify indispensable features of the claimed subject matter, nor is it intended for use as an aid in limiting the scope of the claimed subject matter.

The present disclosure introduces an apparatus that includes a wearable electronic device and a processing device. The wearable electronic device is to be worn by a human at a worksite. The wearable electronic device includes a sensor to detect a physical action and/or experience of the human, and an output device to output a sensory signal to be perceived by the human. The processing device includes a processor and a memory storing computer program code. The processing device is operable to cause the output device to output the sensory signal based on the detected physical action and/or experience.

The present disclosure also introduces a system that includes multiple wearable electronic devices, a database, and a processing device. The wearable electronic devices are to be worn by humans at worksites. Each wearable electronic device includes a sensor to detect a human physical action and/or experience and an output device to output a sensory signal for human perception. The sensory signal is based on at least one of the detected physical actions and/or experiences. The processing device includes a processor and a memory storing computer program code. The processing device is communicatively connected with the wearable electronic devices and the database. The processing device is operable to record the detected physical actions and/or experiences to the database in association with information indicative of types of worksite events performed and/or experienced by the humans that correspond to the detected physical actions and/or experiences. The processing device is also operable to compare a subsequent human physical action and/or experience during a corresponding subsequent worksite event, as detected by a sensor of one of the wearable electronic devices, to the recorded physical actions and/or experiences. The processing device is also operable to determine the type of the subsequent worksite event based on the comparison.

The present disclosure also introduces a method that includes, while at a wellsite, donning an electronic device that includes or is in wireless communication with a processing device. The processing device includes a processor and a memory storing computer program code. The method also includes performing an action or having an experience while at the wellsite. The performed action or experience is detected by the donned electronic device. The method also includes, while at the wellsite, perceiving a sensory signal output by the donned electronic device. The sensory signal output is caused by the processing device based on the detected action or experience.

The present disclosure also introduces a method that includes outputting a sensory signal to be perceived by a human wearing an electronic device at a worksite. The electronic device includes or is in wireless communication with a processing device. The processing device includes a processor and a memory storing computer program code. The electronic device detects physical actions and/or experiences of the human at the wellsite. The sensory signal output is caused by the processing device based on the detected physical actions and/or experiences.

These and additional aspects of the present disclosure are set forth in the description that follows, and/or may be learned by a person having ordinary skill in the art by reading the materials herein and/or practicing the principles described herein. At least some aspects of the present disclosure may be achieved via means recited in the attached claims.

### BRIEF DESCRIPTION OF THE DRAWINGS

The present disclosure is best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a schematic view of at least a portion of an example implementation of apparatus according to one or more aspects of the present disclosure.

FIG. 2 is a schematic view of at least a portion of an example implementation of apparatus according to one or more aspects of the present disclosure.

FIG. 3 is a schematic view of at least a portion of an example implementation of apparatus according to one or more aspects of the present disclosure.

FIG. 4 is a schematic view of at least a portion of an example implementation of apparatus according to one or more aspects of the present disclosure.

FIG. 5 is a schematic view of at least a portion of an example implementation of apparatus according to one or more aspects of the present disclosure.

FIG. 6 is a schematic view of at least a portion of an example implementation of apparatus according to one or more aspects of the present disclosure.

FIG. 7 is a schematic view of at least a portion of an example implementation of apparatus according to one or more aspects of the present disclosure.

FIG. 8 is a schematic view of at least a portion of an example implementation of apparatus according to one or more aspects of the present disclosure.

FIG. 9 is a schematic view of at least a portion of an example implementation of apparatus according to one or more aspects of the present disclosure.

FIG. 10 is a schematic view of at least a portion of an example implementation of apparatus according to one or more aspects of the present disclosure.

FIG. 11 is a schematic view of at least a portion of an example implementation of apparatus during various stages of operations according to one or more aspects of the present disclosure.

FIG. 12 is a schematic view of at least a portion of an example implementation of apparatus during various stages of operations according to one or more aspects of the present disclosure.

FIG. 13 is a schematic view of at least a portion of an example implementation of apparatus during various stages of operations according to one or more aspects of the present disclosure.

FIG. 14 is a schematic view of at least a portion of an example implementation of apparatus during various stages of operations according to one or more aspects of the present disclosure.

FIG. 15 is a geometric shape related to one or more aspects of the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for simplicity and clarity, and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

Systems and methods (e.g., processes, operations) according to one or more aspects of the present disclosure may be utilized or otherwise implemented in association with an automated well construction system at an oil and gas wellsite, such as for constructing a wellbore to obtain hydrocarbons (e.g., oil and/or gas) from a subterranean formation. However, one or more aspects of the present disclosure may be utilized or otherwise implemented in association with other automated systems in the oil and gas industry and other industries. For example, one or more aspects of the present disclosure may be implemented in association with wellsite systems for performing fracturing, cementing, acidizing, chemical injecting, and/or water jet cutting operations, among other examples. One or more aspects of the present disclosure may also be implemented in association with mining sites, building construction sites, manufacturing facilities, maintenance (e.g., repair) facilities, and/or other worksites where automated machines or equipment are utilized.

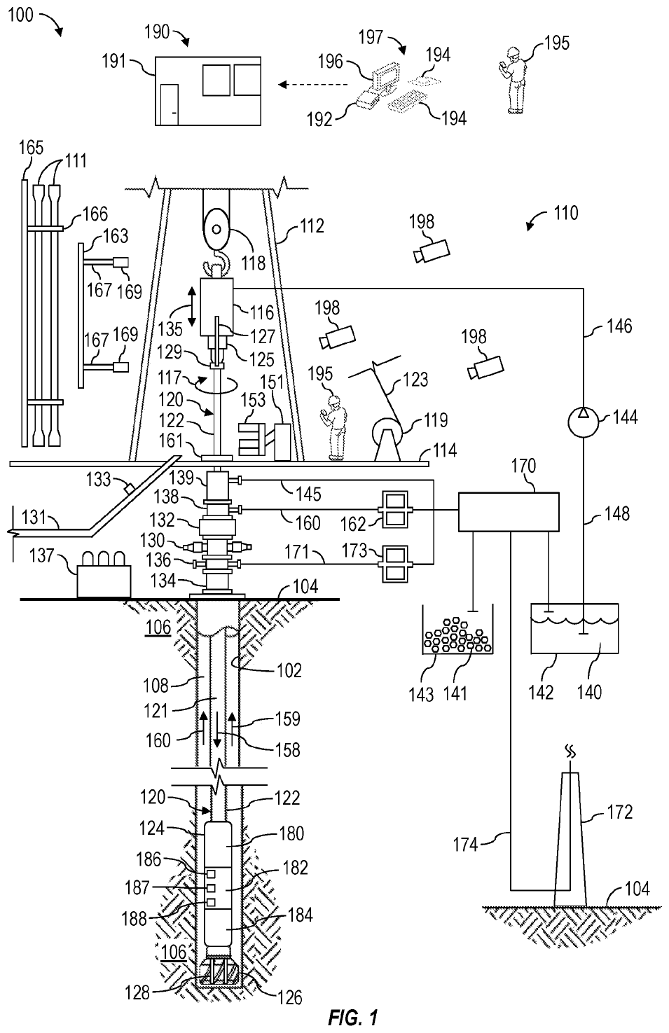

FIG. 1 is a schematic view of at least a portion of an example implementation of a well construction system 100 according to one or more aspects of the present disclosure. The well construction system 100 represents an example environment in which one or more aspects of the present disclosure described below may be implemented. Although the well construction system 100 is depicted as an onshore implementation, the aspects described below are also applicable to offshore implementations.

The well construction system 100 is depicted in relation to a wellbore 102 formed by rotary and/or directional drilling from a wellsite surface 104 and extending into a subterranean formation 106. The well construction system 100 includes surface equipment 110 located at the wellsite surface 104 and a drill string 120 suspended within the wellbore 102. The surface equipment 110 may include a mast, a derrick, and/or another support structure 112 disposed over a rig floor 114. The drill string 120 may be suspended within the wellbore 102 from the support structure 112. The support structure 112 and the rig floor 114 are collectively supported over the wellbore 102 by legs and/or other support structures (not shown).

The drill string 120 may comprise a bottom-hole assembly (BHA) 124 and means 122 for conveying the BHA 124 within the wellbore 102. The conveyance means 122 may comprise drill pipe, heavy-weight drill pipe (HWDP), wired drill pipe (WDP), tough logging condition (TLC) pipe, coiled tubing, and/or other means for conveying the BHA 124 within the wellbore 102. A downhole end of the BHA 124 may include or be coupled to a drill bit 126. Rotation of the drill bit 126 and the weight of the drill string 120 collectively operate to form the wellbore 102. The drill bit 126 may be rotated from the wellsite surface 104 and/or via a downhole mud motor (not shown) connected with the drill bit 126.

The BHA 124 may also include various downhole tools 180, 182, 184. One or more of such downhole tools 180, 182, 184 may be or comprise an acoustic tool, a density tool, a directional drilling tool, an electromagnetic (EM) tool, a formation sampling tool, a formation testing tool, a gravity tool, a monitoring tool, a neutron tool, a nuclear tool, a photoelectric factor tool, a porosity tool, a reservoir characterization tool, a resistivity tool, a rotational speed sensing tool, a sampling-while-drilling (SWD) tool, a seismic tool, a surveying tool, a torsion sensing tool, and/or other measuring-while-drilling (MWD) or logging-while-drilling (LWD) tools.

One or more of the downhole tools 180, 182, 184 may be or comprise an MWD or LWD tool comprising a sensor package 186 operable for the acquisition of measurement data pertaining to the BHA 124, the wellbore 102, and/or the formation 106. One or more of the downhole tools 180, 182, 184 and/or another portion of the BHA 124 may also comprise a telemetry device 187 operable for communication with the surface equipment 110, such as via mud-pulse telemetry. One or more of the downhole tools 180, 182, 184 and/or another portion of the BHA 124 may also comprise a downhole processing device 188 operable to receive, process, and/or store information received from the surface equipment 110, the sensor package 186, and/or other portions of the BHA 124. The processing device 188 may also store executable computer programs (e.g., program code instructions), including for implementing one or more aspects of the operations described herein.

The support structure 112 may support a driver, such as a top drive 116, operable to connect (perhaps indirectly) with an uphole end of the conveyance means 122, and to impart rotary motion 117 and vertical motion 135 to the drill string 120 and the drill bit 126. However, another driver, such as a kelly and rotary table (neither shown), may be utilized instead of or in addition to the top drive 116 to impart the rotary motion 117. The top drive 116 and the connected drill string 120 may be suspended from the support structure 112 via hoisting equipment, which may include a traveling block 118, a crown block (not shown), and a draw works 119 storing a support cable or line 123. The crown block may be connected to or otherwise supported by the support structure 112, and the traveling block 118 may be coupled with the top drive 116, such as via a hook. The draw works 119 may be mounted on or otherwise supported by the rig floor 114. The crown block and traveling block 118 comprise pulleys or sheaves around which the support line 123 is reeved to operatively connect the crown block, the traveling block 118, and the draw works 119 (and perhaps an anchor). The draw works 119 may thus selectively impart tension to the support line 123 to lift and lower the top drive 116, resulting in the vertical motion 135. The draw works 119 may comprise a drum, a frame, and a prime mover (e.g., an engine or motor) (not shown) operable to drive the drum to rotate and reel in the support line 123, causing the traveling block 118 and the top drive 116 to move upward. The draw works 119 may be operable to release the support line 123 via a controlled rotation of the drum, causing the traveling block 118 and the top drive 116 to move downward.

The top drive 116 may comprise a grabber, a swivel (neither shown), a tubular handling assembly links 127 terminating with an elevator 129, and a drive shaft 125 operatively connected with a prime mover (not shown), such as via a gear box or transmission (not shown). The drill string 120 may be mechanically coupled to the drive shaft 125 with or without a sub saver between the drill string 120 and the drive shaft 125. The prime mover may be selectively operated to rotate the drive shaft 125 and the drill string 120 coupled with the drive shaft 125. Hence, during drilling operations, the top drive 116 in conjunction with operation of the draw works 119 may advance the drill string 120 into the formation 106 to form the wellbore 102. The tubular handling assembly links 127 and the elevator 129 of the top drive 116 may handle tubulars (e.g., drill pipes, drill collars, casing joints, etc.) that are not mechanically coupled to the drive shaft 125. For example, when the drill string 120 is being tripped into or out of the wellbore 102, the elevator 129 may grasp the tubulars of the drill string 120 such that the tubulars may be raised and/or lowered via the hoisting equipment mechanically coupled to the top drive 116. The grabber may include a clamp that clamps onto a tubular when making up and/or breaking out a connection of a tubular with the drive shaft 125. The top drive 116 may have a guide system (not shown), such as rollers that track up and down a guide rail on the support structure 112. The guide system may aid in keeping the top drive 116 aligned with the wellbore 102, and in preventing the top drive 116 from rotating during drilling by transferring reactive torque to the support structure 112.

The well construction system 100 may further include a well control system for maintaining well pressure control. For example, the drill string 120 may be conveyed within the wellbore 102 through various blowout preventer (BOP) equipment disposed at the wellsite surface 104 on top of the wellbore 102 and perhaps below the rig floor 114. The BOP equipment may be operable to control pressure within the wellbore 102 via a series of pressure barriers (e.g., rams) between the wellbore 102 and the wellsite surface 104. The BOP equipment may include a BOP stack 130, an annular preventer 132, and/or a rotating control device (RCD) 138 mounted above the annular preventer 132. The BOP equipment 130, 132, 138 may be mounted on top of a wellhead 134. The well control system may further include a BOP control unit 137 (i.e., a BOP closing unit) operatively connected with the BOP equipment 130, 132, 138 and operable to actuate, drive, operate or otherwise control the BOP equipment 130, 132, 138. The BOP control unit 137 may be or comprise a hydraulic fluid power unit fluidly connected with the BOP equipment 130, 132, 138 and selectively operable to hydraulically drive various portions (e.g., rams, valves, seals) of the BOP equipment 130, 132, 138.

The well construction system 100 may further include a drilling fluid circulation system operable to circulate fluids between the surface equipment 110 and the drill bit 126 during drilling and other operations. For example, the drilling fluid circulation system may be operable to inject a drilling fluid from the wellsite surface 104 into the wellbore 102 via an internal fluid passage 121 extending longitudinally through the drill string 120. The drilling fluid circulation system may comprise a pit, a tank, and/or other fluid container 142 holding the drilling fluid (i.e., mud) 140, and a pump 144 operable to move the drilling fluid 140 from the container 142 into the fluid passage 121 of the drill string 120 via a fluid conduit 146 extending from the pump 144 to the top drive 116 and an internal passage extending through the top drive 116. The fluid conduit 146 may comprise one or more of a pump discharge line, a stand pipe, a rotary hose, and a gooseneck (not shown) connected with a fluid inlet of the top drive 116. The pump 144 and the container 142 may be fluidly connected by a fluid conduit 148, such as a suction line.

During drilling operations, the drilling fluid may continue to flow downhole through the internal passage 121 of the drill string 120, as indicated by directional arrow 158. The drilling fluid may exit the BHA 124 via ports 128 in the drill bit 126 and then circulate uphole through an annular space 108 (“annulus”) of the wellbore 102 defined between an exterior of the drill string 120 and the wall of the wellbore 102, such flow being indicated by directional arrows 159. In this manner, the drilling fluid lubricates the drill bit 126 and carries formation cuttings uphole to the wellsite surface 104. The returning drilling fluid may exit the annulus 108 via a bell nipple 139, an RCD 138, and/or a ported adapter 136 (e.g., a spool, a wing valve, etc.) located below one or more portions of the BOP stack 130.

The drilling fluid exiting the annulus 108 via the bell nipple 139 may be directed toward drilling fluid reconditioning equipment 170 via a fluid conduit 145 (e.g., gravity return line) to be cleaned and/or reconditioned, as described below, prior to being returned to the container 142 for recirculation. The drilling fluid exiting the annulus 108 via the RCD 138 may be directed into a fluid conduit 160 (e.g., a drilling pressure control line), and may pass through various wellsite equipment fluidly connected along the conduit 160 prior to being returned to the container 142 for recirculation. For example, the drilling fluid may pass through a choke manifold 162 (e.g., a drilling pressure control choke manifold) and then through the drilling fluid reconditioning equipment 170. The choke manifold 162 may include at least one choke and a plurality of fluid valves (neither shown) collectively operable to control the flow through and out of the choke manifold 162. Backpressure may be applied to the annulus 108 by variably restricting flow of the drilling fluid or other fluids flowing through the choke manifold 162. The greater the restriction to flow through the choke manifold 162, the greater the backpressure applied to the annulus 108. The drilling fluid exiting the annulus 108 via the ported adapter 136 may be directed into a fluid conduit 171 (e.g., rig choke line), and may pass through various equipment fluidly connected along the conduit 171 prior to being returned to the container 142 for recirculation. For example, the drilling fluid may pass through a choke manifold 173 (e.g., a rig choke manifold, well control choke manifold) and then through the drilling fluid reconditioning equipment 170. The choke manifold 173 may include at least one choke and a plurality of fluid valves (neither shown) collectively operable to control the flow through the choke manifold 173. Backpressure may be applied to the annulus 108 by variably restricting flow of the drilling fluid or other fluids flowing through the choke manifold 173.

Before being returned to the container 142, the drilling fluid returning to the wellsite surface 104 may be cleaned and/or reconditioned via drilling fluid reconditioning equipment 170, which may include one or more of liquid gas separators, shale shakers, centrifuges, and other drilling fluid cleaning equipment. The liquid gas separators may remove formation gasses entrained in the drilling fluid discharged from the wellbore 102 and the shale shakers may separate and remove solid particles 141 (e.g., drill cuttings) from the drilling fluid. The drilling fluid reconditioning equipment 170 may further comprise equipment operable to remove additional gas and finer formation cuttings from the drilling fluid and/or modify physical properties or characteristics (e.g., rheology) of the drilling fluid. For example, the drilling fluid reconditioning equipment 170 may include a degasser, a desander, a desilter, a mud cleaner, and/or a decanter, among other examples. Intermediate tanks/containers (not shown) may be utilized to hold the drilling fluid while the drilling fluid progresses through the various stages or portions of the drilling fluid reconditioning equipment 170. The cleaned/reconditioned drilling fluid may be transferred to the fluid container 142, the solid particles 141 removed from the drilling fluid may be transferred to a solids container 143 (e.g., a reserve pit), and/or the removed gas may be transferred to a flare stack 172 via a conduit 174 (e.g., a flare line) to be burned or to a container (not shown) for storage and removal from the wellsite.

The surface equipment 110 may include tubular handling equipment operable to store, move, connect, and disconnect tubulars (e.g., drill pipes) to assemble and disassemble the conveyance means 122 of the drill string 120 during drilling operations. For example, a catwalk 131 may be utilized to convey tubulars from a ground level, such as along the wellsite surface 104, to the rig floor 114, permitting the tubular handling assembly links 127 to grab and lift the tubulars above the wellbore 102 for connection with previously deployed tubulars. The catwalk 131 may have a horizontal portion and an inclined portion that extends between the horizontal portion and the rig floor 114. The catwalk 131 may comprise a skate 133 movable along a groove (not shown) extending longitudinally along the horizontal and inclined portions of the catwalk 131. The skate 133 may be operable to convey (e.g., push) the tubulars along the catwalk 131 to the rig floor 114. The skate 133 may be driven along the groove by a drive system (not shown), such as a pulley system or a hydraulic system. Additionally, one or more racks (not shown) may adjoin the horizontal portion of the catwalk 131. The racks may have a spinner unit for transferring tubulars to the groove of the catwalk 131.

An iron roughneck 151 may be positioned on the rig floor 114. The iron roughneck 151 may comprise a torqueing portion 153, such as may include a spinner and a torque wrench comprising a lower tong and an upper tong. The torqueing portion 153 of the iron roughneck 151 may be moveable toward and at least partially around the drill string 120, such as may permit the iron roughneck 151 to make up and break out connections of the drill string 120. The torqueing portion 153 may also be moveable away from the drill string 120, such as may permit the iron roughneck 151 to move clear of the drill string 120 during drilling operations. The spinner of the iron roughneck 151 may be utilized to apply low torque to make up and break out threaded connections between tubulars of the drill string 120, and the torque wrench may be utilized to apply a higher torque to tighten and loosen the threaded connections.

Reciprocating slips 161 may be located on the rig floor 114, such as may accommodate therethrough the downhole tubulars during make up and break out operations and during the drilling operations. The reciprocating slips 161 may be in an open position during drilling operations to permit advancement of the drill string 120 therethrough, and in a closed position to clamp an upper end of the conveyance means 122 (e.g., assembled tubulars) to thereby suspend and prevent advancement of the drill string 120 within the wellbore 102, such as during the make up and break out operations.

During drilling operations, the hoisting equipment lowers the drill string 120 while the top drive 116 rotates the drill string 120 to advance the drill string 120 downward within the wellbore 102 and into the formation 106. During the advancement of the drill string 120, the reciprocating slips 161 are in an open position, and the iron roughneck 151 is moved away or is otherwise clear of the drill string 120. When the upper portion of the tubular in the drill string 120 that is made up to the drive shaft 125 is near the reciprocating slips 161 and/or the rig floor 114, the top drive 116 ceases rotating and the reciprocating slips 161 close to clamp the tubular made up to the drive shaft 125. The grabber (not shown) of the top drive 116 then clamps the upper portion of the tubular made up to the drive shaft 125, and the drive shaft 125 rotates in a direction reverse from the drilling rotation to break out the connection between the drive shaft 125 and the made up tubular. The grabber of the top drive 116 may then release the tubular of the drill string 120.

Multiple tubulars may be loaded on the rack of the catwalk 131 and individual tubulars (or stands of two or three tubulars) may be transferred from the rack to the groove in the catwalk 131, such as by the spinner unit. The tubular positioned in the groove may be conveyed along the groove by the skate 133 until an end of the tubular projects above the rig floor 114. The elevator 129 of the top drive 116 then grasps the protruding end, and the draw works 119 is operated to lift the top drive 116, the elevator 129, and the new tubular.

The hoisting equipment then raises the top drive 116, the elevator 129, and the tubular until the tubular is aligned with the upper portion of the drill string 120 clamped by the slips 161. The iron roughneck 151 is moved toward the drill string 120, and the lower tong of the torqueing portion 153 clamps onto the upper portion of the drill string 120. The spinning system rotates the new tubular (e.g., a threaded male end) into the upper portion of the drill string 120 (e.g., a threaded female end). The upper tong then clamps onto the new tubular and rotates with high torque to complete making up the connection with the drill string 120. In this manner, the new tubular becomes part of the drill string 120. The iron roughneck 151 then releases and moves clear of the drill string 120.

The grabber of the top drive 116 may then clamp onto the drill string 120. The drive shaft 125 (e.g., a threaded male end) is brought into contact with the drill string 120 (e.g., a threaded female end) and rotated to make up a connection between the drill string 120 and the drive shaft 125. The grabber then releases the drill string 120, and the reciprocating slips 161 are moved to the open position. The drilling operations may then resume.

The tubular handling equipment may further include a pipe handling manipulator (PHM) 163 disposed in association with a fingerboard 165. Although the PHM 163 and the fingerboard 165 are shown separate and distinct from the support structure 112, each of the PHM 163 and the fingerboard 165 may be supported by or otherwise connected with the support structure 112 or another portion of the well construction system 100. The fingerboard 165 provides storage (e.g., temporary storage) of tubulars (or stands of two or three tubulars) 111 during various operations, such as during and between tripping out and tripping in the drill string 120. The fingerboard 165 may comprise a rack 166 defining a plurality of slots configured to support or otherwise hold the tubulars 111. The PHM 163 may be operable to transfer the tubulars 111 between the fingerboard 165 and the drill string 120 (i.e., space above the suspended drill string 120). For example, the PHM 163 may include arms 167 terminating with clamps 169, such as may be operable to grasp and/or clamp onto one of the tubulars 111. The arms 167 of the PHM 163 may extend and retract, and/or at least a portion of the PHM 163 may be rotatable and/or movable toward and away from the drill string 120, such as may permit the PHM 163 to transfer the tubular 111 between the fingerboard 165 and the drill string 120.

To trip out the drill string 120, the top drive 116 is raised, the reciprocating slips 161 are closed around the drill string 120, and the elevator 129 is closed around the drill string 120. The grabber of the top drive 116 clamps the upper portion of the tubular made up to the drive shaft 125. The drive shaft 125 then rotates in a direction reverse from the drilling rotation to break out the connection between the drive shaft 125 and the drill string 120. The grabber of the top drive 116 then releases the tubular of the drill string 120, and the drill string 120 is suspended by (at least in part) the elevator 129. The iron roughneck 151 is moved toward the drill string 120. The lower tong clamps onto a lower tubular below a connection of the drill string 120, and the upper tong clamps onto an upper tubular above that connection. The upper tong then rotates the upper tubular to provide a high torque to break out the connection between the upper and lower tubulars. The spinning system then rotates the upper tubular to separate the upper and lower tubulars, such that the upper tubular is suspended above the rig floor 114 by the elevator 129. The iron roughneck 151 then releases the drill string 120 and moves clear of the drill string 120.

The PHM 163 may then move toward the drill string 120 to grasp the tubular suspended from the elevator 129. The elevator 129 then opens to release the tubular. The PHM 163 then moves away from the drill string 120 while grasping the tubular with the clamps 169, places the tubular in the fingerboard 165, and releases the tubular for storage in the fingerboard 165. This process is repeated until the intended length of drill string 120 is removed from the wellbore 102.

The surface equipment 110 of the well construction system 100 may also comprise a control center 190 from which various portions of the well construction system 100, such as the top drive 116, the hoisting system, the tubular handling system, the drilling fluid circulation system, the well control system, the BHA 124, among other examples, may be monitored and controlled. The control center 190 may be located on the rig floor 114 or another location of the well construction system 100, such as the wellsite surface 104. The control center 190 may comprise a facility 191 (e.g., a room, a cabin, a trailer, etc.) containing a control workstation 197, which may be operated by a human wellsite operator 195 to monitor and control various wellsite equipment or portions of the well construction system 100. The control workstation 197 may comprise or be communicatively connected with a processing device 192 (e.g., a controller, a computer, etc.), such as may be operable to receive, process, and output information to monitor operations of and provide control to one or more portions of the well construction system 100. For example, the processing device 192 may be communicatively connected with the various surface and downhole equipment described herein, and may be operable to receive signals from and transmit signals to such equipment to perform various operations described herein. The processing device 192 may store executable program code, instructions, and/or operational parameters or set-points, including for implementing one or more aspects of methods and operations described herein. The processing device 192 may be located within and/or outside of the facility 191.

The control workstation 197 may be operable for entering or otherwise communicating control commands to the processing device 192 by the wellsite operator 195, and for displaying or otherwise communicating information from the processing device 192 to the wellsite operator 195. The control workstation 197 may comprise a plurality of human-machine interface (HMI) devices, including one or more input devices 194 (e.g., a keyboard, a mouse, a joystick, a touchscreen, etc.) and one or more output devices 196 (e.g., a video monitor, a touchscreen, a printer, audio speakers, etc.). Communication between the processing device 192, the input and output devices 194, 196, and the various wellsite equipment may be via wired and/or wireless communication means. However, for clarity and ease of understanding, such communication means are not depicted, and a person having ordinary skill in the art will appreciate that such communication means are within the scope of the present disclosure.

Well construction systems within the scope of the present disclosure may include more or fewer components than as described above and depicted in FIG. 1. Additionally, various equipment and/or subsystems of the well construction system 100 shown in FIG. 1 may include more or fewer components than as described above and depicted in FIG. 1. For example, various engines, motors, hydraulics, actuators, valves, and/or other components not explicitly described herein may be included in the well construction system 100, and are within the scope of the present disclosure.

The well construction system 100 also includes stationary and/or mobile video cameras 198 disposed or utilized at various locations within the well construction system 100. The video cameras 198 capture videos of various portions, equipment, or subsystems of the well construction system 100, and perhaps the wellsite operators 195 and the actions they perform, during or otherwise in association with the wellsite operations, including while performing repairs to the well construction system 100 during a breakdown. For example, the video cameras 198 may capture digital images (or video frames) of the entire well construction system 100 and/or specific portions of the well construction system 100, such as the top drive 116, the iron roughneck 151, the PHM 163, the fingerboard 165, and/or the catwalk 131, among other examples. The video cameras 198 generate corresponding video signals (i.e., feeds) comprising or otherwise indicative of the captured digital images. The video cameras 198 may be in signal communication with the processing device 192, such as may permit the video signals to be processed and transmitted to the control workstation 197 and, thus, permit the wellsite operators 195 to view various portions or components of the well construction system 100 on one or more of the output devices 196. The processing device 192 or another portion of the control workstation 197 may be operable to record the video signals generated by the video cameras 198.

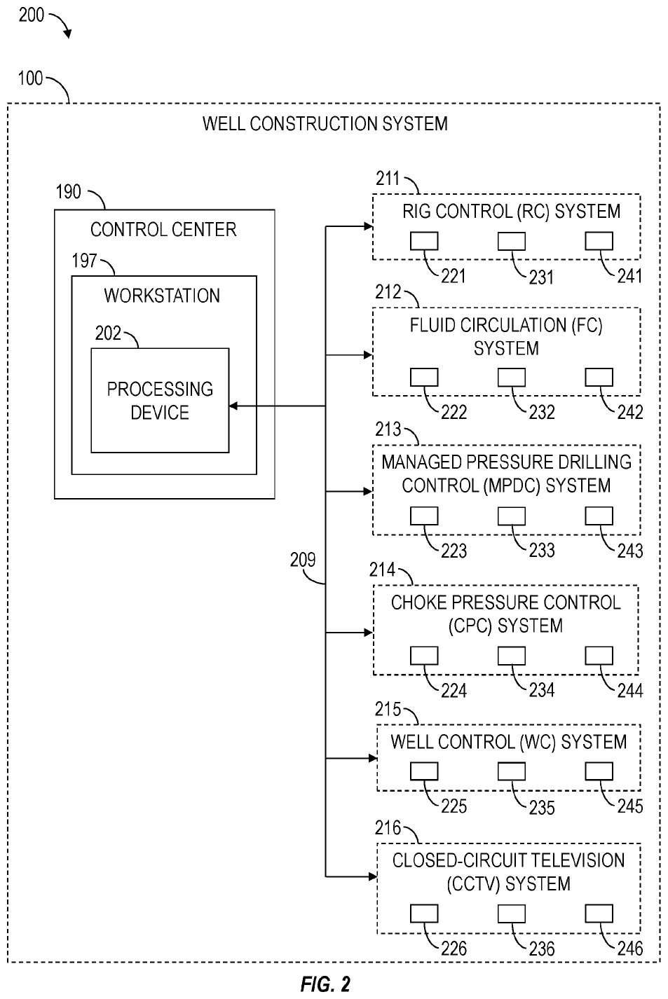

The present disclosure further provides various implementations of systems and/or methods for controlling one or more portions of the well construction system 100. FIG. 2 is a schematic view of at least a portion of an example implementation of a monitoring and control system 200 for monitoring and controlling various equipment, portions, and subsystems of the well construction system 100 according to one or more aspects of the present disclosure. The following description refers to FIGS. 1 and 2, collectively.

The control system 200 may be in real-time communication with and utilized to monitor and/or control various portions, components, and equipment of the well construction system 100 described herein. The equipment of the well construction system 100 may be grouped into several subsystems, each operable to perform a corresponding operation and/or a portion of the well construction operations described herein. The subsystems may include a rig control (RC) system 211, a fluid circulation (FC) system 212, a managed pressure drilling control (MPDC) system 213, a choke pressure control (CPC) system 214, a well pressure control (WC) system 215, and a closed-circuit television (CCTV) system 216. The control workstation 197 may be utilized to monitor, configure, control, and/or otherwise operate one or more of the well construction subsystems 211-216.

The RC system 211 may include the support structure 112, the drill string hoisting system or equipment (e.g., the draw works 119 and the top drive 116), drill string drivers (e.g., the top drive 116 and/or the rotary table and kelly), the reciprocating slips 161, the drill pipe handling system or equipment (e.g., the catwalk 131, the PHM 163, the fingerboard 165, and the iron roughneck 151), electrical generators, and other equipment. Accordingly, the RC system 211 may perform power generation and drill pipe handling, hoisting, and rotation operations. The RC system 211 may also serve as a support platform for drilling equipment and staging ground for rig operations, such as connection make up and break out operations described above. The FC system 212 may include the drilling fluid 140, the pumps 144, drilling fluid loading equipment, the drilling fluid reconditioning equipment 170, the flare stack 172, and/or other fluid control equipment. Accordingly, the FC system 212 may perform fluid operations of the well construction system 100. The MPDC system 213 may include the RCD 138, the choke manifold 162, downhole pressure sensors 186, and/or other equipment. The CPC system 214 may comprise the choke manifold 173, and/or other equipment, and the WC system 215 may comprise the BOP equipment 130, 132, 138, the BOP control unit 137, and a BOP control station (not shown) for controlling the BOP control unit 137. The CCTV system 216 may include the video cameras 198 and corresponding actuators (e.g., motors) for moving or otherwise controlling direction of the video cameras 198. The CCTV system 216 may be utilized to capture real-time video of various portions or subsystems 211-215 of the well construction system 100 and display video signals from the video cameras 198 on the video output devices 196 to display in real-time the various portions or subsystems 211-215. Each of the well construction subsystems 211-216 may further comprise various communication equipment (e.g., modems, network interface cards, etc.) and communication conductors (e.g., cables), communicatively connecting the equipment (e.g., sensors and actuators) of each subsystem 211-216 with the control workstation 197 and/or other equipment. Although the wellsite equipment listed above and shown in FIG. 1 is associated with certain wellsite subsystems 211-216, such associations are merely examples that are not intended to limit or prevent such wellsite equipment from being associated with two or more wellsite subsystems 211-216 and/or different wellsite subsystems 211-216.

The control system 200 may also include various local controllers 221-226 associated with corresponding subsystems 211-216 and/or individual pieces of equipment of the well construction system 100. As described above, each well construction subsystem 211-216 includes various wellsite equipment comprising corresponding actuators 241-246 for performing operations of the well construction system 100. Each subsystem 211-216 further includes various sensors 231-236 operable to generate sensor data indicative of operational performance and/or status of the wellsite equipment of each subsystem 211-216. Although the sensors 231-236 and actuators 241-246 are each shown as a single block, it is to be understood that each sensor 231-236 and actuator 241-246 may be or comprise a plurality of sensors and actuators, whereby each actuator performs a corresponding action of a piece of equipment or subsystem 211-216 and each sensor generates corresponding sensor data indicative of the action performed by a corresponding actuator or of other operational parameter of the piece of equipment or subsystem 211-216.

The local controllers 221-226, the sensors 231-236, and the actuators 241-246 may be communicatively connected with a processing device 202. For example, the local controllers may be in communication with the sensors 231-236 and actuators 241-246 of the corresponding subsystems 211-216 via local communication networks (e.g., field buses, not shown) and the processing device 202 may be in communication with the subsystems 211-216 via a communication network 209 (e.g., data bus, a wide-area-network (WAN), a local-area-network (LAN), etc.). The sensor data (e.g., electronic signals, information, and/or measurements, etc.) generated by the sensors 231-236 of the subsystems 211-216 may be made available for use by processing device 202 and/or the local controllers 221-226. Similarly, control commands (e.g., signals, information, etc.) generated by the processing device 202 and/or the local controllers 221-226 may be automatically communicated to the various actuators 241-246 of the subsystems 211-216, perhaps pursuant to predetermined programming, such as to facilitate well construction operations and/or other operations described herein. The processing device 202 may be or comprise the processing device 192 shown in FIG. 1. Accordingly, the processing device 202 may be communicatively connected with or form a portion of the workstation 197 and/or may be at least partially located within the control center 190.

The sensors 231-236 and actuators 241-246 may be monitored and/or controlled by the processing device 202. For example, the processing device 202 may be operable to receive the sensor data from the sensors 231-236 of the wellsite subsystems 211-216 in real-time, and to provide real-time control commands to the actuators 241-246 of the subsystems 211-216 based on the received sensor data. However, certain operations of the actuators 241-246 may be controlled by the local controllers 221-226, which may control the actuators 241-246 based on sensor data received from the sensors 231-236 and/or based on control commands received from the processing device 202.

The processing devices 188, 192, 202, the local controllers 221-226, and other controllers or processing devices of the well construction system 100 may be operable to receive program code instructions and/or sensor data from sensors (e.g., sensors 231-236), process such information, and/or generate control commands to operate controllable equipment (e.g., actuators 241-246) of the well construction system 100. Accordingly, the processing devices 188, 192, 202, the local controllers 221-226, and other controllers or processing devices of the well construction system 100 may individually or collectively be referred to hereinafter as equipment controllers. Equipment controllers within the scope of the present disclosure can include, for example, programmable logic controllers (PLCs), industrial computers (IPCs), personal computers (PCs), soft PLCs, variable frequency drives (VFDs) and/or other controllers or processing devices operable to receive sensor data and/or control commands and cause operation of controllable equipment based on such sensor data and/or control commands.

Various pieces of wellsite equipment described above and shown in FIGS. 1 and 2 may each comprise one or more hydraulic and/or electrical actuators, which when actuated, may cause corresponding components or portions of the piece of equipment to perform intended actions (e.g., work, tasks, movements, operations, etc.). Each such piece of equipment may further comprise a plurality of sensors, whereby one or more sensors may be associated with a corresponding actuator or another component of the piece of equipment and communicatively connected with an equipment controller. Each sensor may be operable to generate sensor data (e.g., electrical sensor signals or measurements) indicative of an operational (e.g., mechanical, physical) status of the corresponding actuator or component, thereby permitting the operational status of the actuator to be monitored by the equipment controller. The sensor data may be utilized by the equipment controller as feedback data, permitting operational control of the piece of equipment and coordination with other equipment. Such sensor data may be indicative of performance of each individual actuator and, collectively, of the entire piece of wellsite equipment.

The present disclosure is further directed to electronic devices supported or carried by, integrated with, or otherwise disposed in association with corresponding wearable articles, such as wristbands, gloves, safety glasses, safety hats, safety vests, overalls, jackets, and other outerwear wearable by humans (e.g., workers or personnel, such as wellsite operators and maintenance personnel). Such wearable electronic devices (i.e., wearable technology) may be operable to augment human activity at a worksite (e.g., wellsite, mining site, building construction site, etc.) or in a remotely located maintenance shop or other facility. The present disclosure is also directed to processes and/or methods of utilizing such wearable electronic devices. For example, the wearable electronic device may be utilized to detect and/or determine attributes associated with the person wearing the wearable electronic device, including the location of the person, the type of activity (e.g., job, task, etc.) being performed by the person, the number of tasks completed by the person, and the time taken to perform each task by the person, among other examples. A report listing the determined attributes and/or other information based on such attributes may then be automatically generated for a given activity or activities. The determined attributes and/or other information may then be utilized to optimize or otherwise plan maintenance activities by setting benchmarks, estimating amount of time to complete each activity, and estimating the amount of time to have the equipment available. The wearable electronic devices may also or instead be utilized to improve safety of the personnel wearing the wearable electronic devices at the worksite. For example, the wearable electronic devices may detect and/or determine if personnel at the worksite are performing unsafe actions. The wearable electronic devices can also be utilized as a feedback mechanism to identify, reduce, and/or remove safety risks at the worksite, improve situational awareness at the worksite, and/or otherwise make the worksite safer. The wearable electronic devices may direct the personnel to perform certain actions in case of an emergency or otherwise to reduce the chances of the personnel being injured.

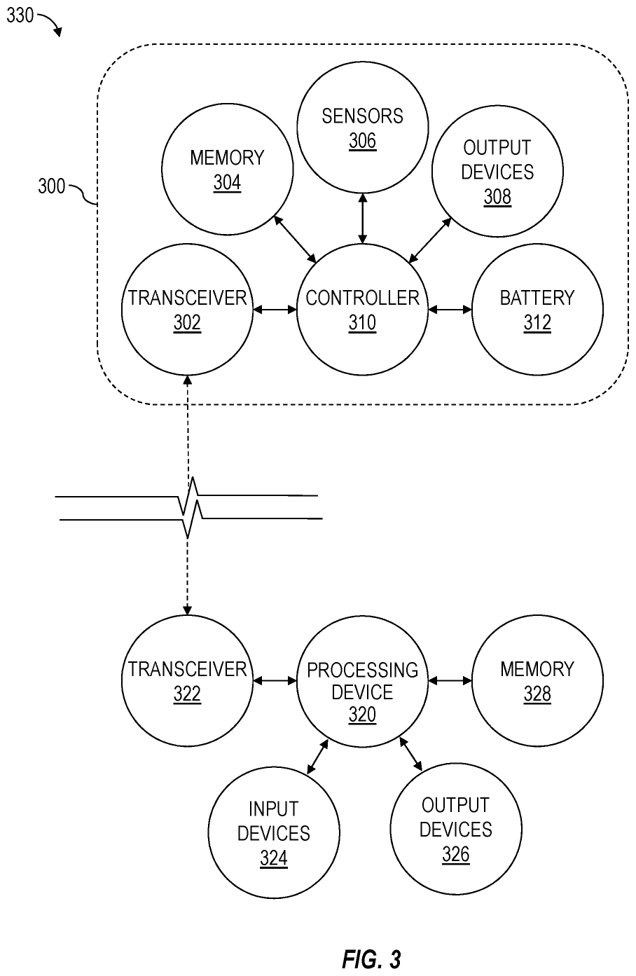

FIG. 3 is a schematic view of at least a portion of an example implementation of a wearable electronic device 300 according to one or more aspects of the present disclosure. The wearable electronic device 300 may be supported by, carried by, integrated with, or otherwise disposed in association with a corresponding article wearable by a human worker (i.e., a person) at a worksite. For example, the wearable electronic device 300 may be disposed in association with a wristband, gloves, safety glasses, safety hat, safety vest, overalls, jacket, and other outerwear worn by the worker.

The wearable electronic device 300 may comprise one or more sensors 306 operable to detect physical actions (e.g., movements, motions) performed and/or experienced by the worker. The sensors 306 may be, comprise, or be implemented by a video camera, a microphone, an accelerometer, an inertial measurement unit, a GPS signal receiver, and/or a position locator among other examples. The camera may be operable to capture digital video and/or images of the worker and/or objects (e.g., equipment) with which the worker interacts. The microphone may be operable to capture the worker's voice and sounds generated by the objects with which the worker interacts. The accelerometer may be operable to detect movements and/or forces exerted or experienced by the worker. The inertial measurement unit (IMU) may be operable to measure or otherwise detect specific force, angular rate, and/or position performed or experienced by the worker. The GPS signal receiver may be operable to receive or acquire location information from a GPS satellite. The GPS signal receiver, the IMU, the position locator, and/or another feature of the wearable electronic device 300 may utilize location information to determine time-stamped geographical location of the wearable electronic device 300 and, thus, the associated (e.g., co-located) worker wearing the wearable electronic device 300. Each sensor 306 may be operable to generate corresponding sensor data (e.g., sensor signals or information) indicative of the detected physical actions performed and/or experienced by the worker.

The wearable electronic device 300 may further comprise one or more output devices 308 operable to output sensory signals to be perceived (e.g., seen, heard, felt) by the worker. The sensory signals may be indicative of physical movements or actions to be performed by the worker. For example, the sensory signals may indicate to the worker to finish work, to leave an area, to walk to a different location, to walk in a certain direction, and to become aware of a specific object and/or general surroundings, among other examples. The output devices 308 may be or comprise one or more light emitting devices (e.g., light emitting diodes (LEDs)) operable to output visual (e.g., light) signals to be seen by the worker. The light emitting devices may be operable to selectively output light having different colors and/or at different frequencies or intervals. The output devices 308 may be or comprise audio emitting devices (e.g., speakers) operable to output audio (e.g., sound, voice) signals to be heard by the worker. The audio emitting devices may be operable to selectively output sounds having different pitch (i.e., frequency) and volume, and/or at different intervals. The output devices 308 may be or comprise vibration emitting devices (e.g., piezoelectric actuators) operable to output vibration (e.g., force) signals to be felt by the worker. The vibration emitting devices may be operable to selectively output vibrations having different amplitudes and frequencies, and/or at different intervals.

The wearable electronic device 300 may also comprise a transceiver 302 operable to transmit and/or receive information (e.g., sensor data, control commands) via a wireless communication network, such as Wi-Fi, a mobile telecommunication cellular network, or a satellite communication network. The transceiver 302 may be or comprise a Wi-Fi transceiver, a very small aperture terminal (VSAT), a cellular network transceiver, a satellite transceiver, and/or another communication device operable to communicate via a wireless communication network. The wearable electronic device 300 may also comprise a memory device 304 (e.g., flash memory) operable to store electronic information (e.g., sensor data, control commands).

The wearable electronic device 300 may also comprise a controller 310 in communication with the devices 302, 304, 306, 308. The controller 310 may be or comprise a processing device having a processor and a memory device for storing executable computer program code, such as may include machine-readable coded instructions that, when executed by the processor, may cause the controller 310 to perform or to cause to be performed at least portions of methods and processes described herein. The controller 310 may be operable to cause the output devices 308 to output the sensory signals based on the physical actions performed and/or experienced by the worker detected by the sensors 306. For example, the controller 310 may be operable to receive the sensor data generated by the sensors 306, process the sensor data, and generate or otherwise output control commands to the output devices 308 to cause the output devices 308 to output the sensory signals based on the sensor data. The controller 310 may be operable to save the sensor data, the control commands, and/or other processed data on the memory device 304. The memory device 304 may also be utilized to run edge analytics on the stored information. The processing of the sensor data by the processor of the controller 310 may include various data analysis techniques to detect or determine actions performed by the worker. The wearable electronic device 300 may comprise a local energy storage device, such as a battery 312, which may supply the components 302, 306, 308, 310 of the wearable electronic device 300 with electrical power.

The components 302, 306, 308, 310, 312 of the wearable electronic device 300 may be integrated as a single member, device, or unit (e.g., contained within a single housing). However, one or more of the components 302, 306, 308, 310, 312 of the wearable electronic device 300 may be physically separated from, but operatively connected with, the other of the components 302, 306, 308, 310, 312. For example, the various sensors 306 and/or output devices 308 may be disjoined from the transceiver 302, the controller 310, and/or the battery 312 when disposed in association with a wearable article. However, the components 302, 306, 308, 310, 312 may be communicatively and/or electrically connected (e.g., via electrical wires) together.

Processing of the sensor data and/or generation of the control commands may also or instead be performed by a remote processing device 320 located at a remote location from the wearable electronic device 300. The processing device 320 may be communicatively connected with a memory device 328 (e.g., flash memory) operable to store electronic information (e.g., sensor data, control commands). The remote processing device 320 may be communicatively connected (e.g., via a wired connection) with a transceiver 322, which in turn, may be communicatively connected with the transceiver 302 via a wireless communication network, such as a Wi-Fi network, a mobile telecommunication cellular network, and/or a satellite communication network. The remote processing device 320 may be or comprise a processing device having a processor and a memory device for storing executable computer program code, such as may include machine-readable coded instructions that, when executed by the processor, may cause the remote processing device 320 to perform or to cause to be performed at least portions of methods and processes described herein. The remote processing device 320 may be operable to receive the sensor data generated by the sensors 306 via the transceivers 302, 322, process the sensor data, and generate or otherwise output control commands to the output devices 308 via the transceivers 302, 322 to cause the output devices 308 to output sensory signals based on the sensor data. The processing device 320 may be operable to save the sensor data, the control commands, and/or other processed data on the memory device 328. The processing of the sensor data by the processor of the remote processing device 320 may include various data analysis techniques to detect or determine actions performed by the worker. The processing device 320 may be or form at least a portion of the processing device 192 shown in FIG. 1 and/or the processing device 202 shown in FIG. 2. The wearable electronic device 300, the transceiver 322, and the processing device 320 may collectively be or form at least a portion of a wireless computing system 330.

The processing device 320 may be connected with or comprise one or more input devices 324, such as may permit a worker to enter data and/or commands to the processing device 320. The input devices 324 may be, comprise, or be implemented by a keyboard, a mouse, a touchscreen, a track-pad, a trackball, an isopoint, and/or a voice recognition system, among other examples. The processing device 320 may be connected with or comprise one or more output devices 326, such as may permit the worker to receive information from the processing device 320. The output devices 326 may be, comprise, or be implemented by a video display device (e.g., a liquid crystal display (LCD) or cathode ray tube display (CRT)), a touchscreen, and/or speakers, among other examples.

FIGS. 4-7 are schematic views of example implementations of wearable electronic devices 502, 504, 506, 508 according to one or more aspects of the present disclosure supported or carried by, integrated with, or otherwise disposed in association with a corresponding wearable article. Each article with the associated wearable electronic device 502, 504, 506, 508 may be worn by a human worker at a worksite, such as the wellsite 104 shown in FIG. 1. The wearable electronic devices 502, 504, 506, 508 may each comprise one or more features and/or modes of operation of the wearable electronic device 300 shown in FIG. 3.

FIG. 4 shows the wearable electronic device 502 disposed in association with safety glasses 512. The wearable electronic device 502 may comprise one or more sensors operable to detect physical actions (e.g., movements, motions) performed and/or experienced by the worker wearing the glasses 512. The wearable electronic device 502 may further comprise one or more output devices operable to output sensory signals to be perceived (e.g., seen, heard, felt) by the worker wearing the glasses 512. For example, the output devices may be or comprise light emitting devices 514 (e.g., light emitting diodes (LEDs)) operable to output visual (e.g., light) signals to be seen by the worker and/or an audio speaker 516 operable to output audio signals to be heard by the worker. The light emitting devices 514 may be separate from, but electrically and/or communicatively connected with, the remaining portion of the wearable electronic device 502 comprising one or more of a transceiver, sensors, a controller, and a battery, as shown in FIG. 3.

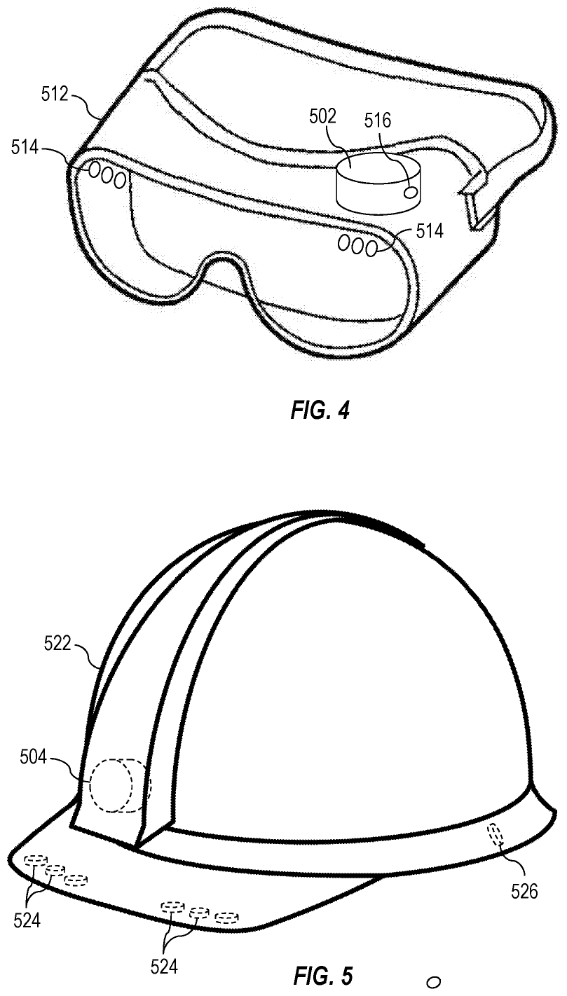

FIG. 5 shows the wearable electronic device 504 disposed in association with a safety hat 522. The wearable electronic device 504 may comprise one or more sensors operable to detect physical actions performed and/or experienced by the worker wearing the hat 522. The wearable electronic device 504 may further comprise one or more output devices operable to output sensory signals to be perceived by the worker wearing the hat 522. For example, the output devices may be or comprise light emitting devices 524 operable to output visual signals to be seen by the worker and/or an audio speaker 526 operable to output audio signals to be heard by the worker. The light emitting devices 524 and/or the audio speaker 626 may be separate from, but electrically and/or communicatively connected with, the remaining portion of the wearable electronic device 504 comprising one or more of a transceiver, sensors, a controller, and a battery, as shown in FIG. 3.

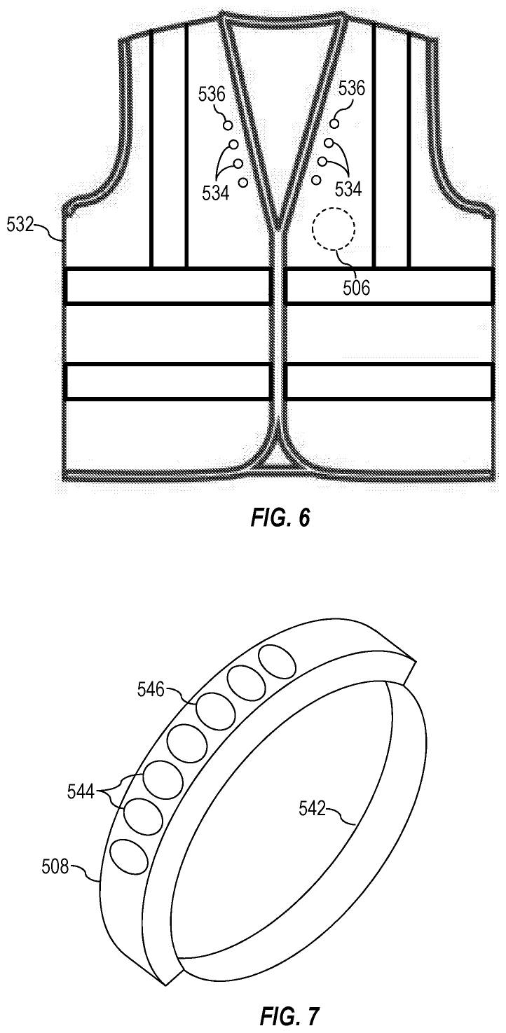

FIG. 6 shows the wearable electronic device 506 disposed in association with a safety vest 532. The wearable electronic device 506 may comprise one or more sensors operable to detect physical actions performed and/or experienced by the worker wearing the vest 522. The wearable electronic device 506 may further comprise one or more output devices operable to output sensory signals to be perceived by the worker wearing the vest 532. For example, the output devices may be or comprise light emitting devices 534 operable to output visual signals to be seen by the worker and/or audio speakers 536 operable to output audio signals to be heard by the worker. The light emitting devices 534 and/or audio speakers 536 may be separate from, but electrically and/or communicatively connected with, the remaining portion of the wearable electronic device 506 comprising one or more of a transceiver, sensors, a controller, and a battery, as shown in FIG. 3.

FIG. 7 shows the wearable electronic device 508 disposed in association with a wrist band 542. The wearable electronic device 508 may comprise one or more sensors operable to detect physical actions performed and/or experienced by the worker wearing the band 542. The wearable electronic device 508 may further comprise one or more output devices operable to output sensory signals to be perceived by the worker wearing the band 542. For example, the output devices may be or comprise light emitting devices 544 operable to output visual signals to be seen by the worker wearing the band 542 and/or an audio speaker 546 operable to output audio signals to be heard by the worker. Although not show in FIG. 7, the wearable electronic device 508 may further comprise one or more of a transceiver, sensors, a controller, and a battery, as shown in FIG. 3.

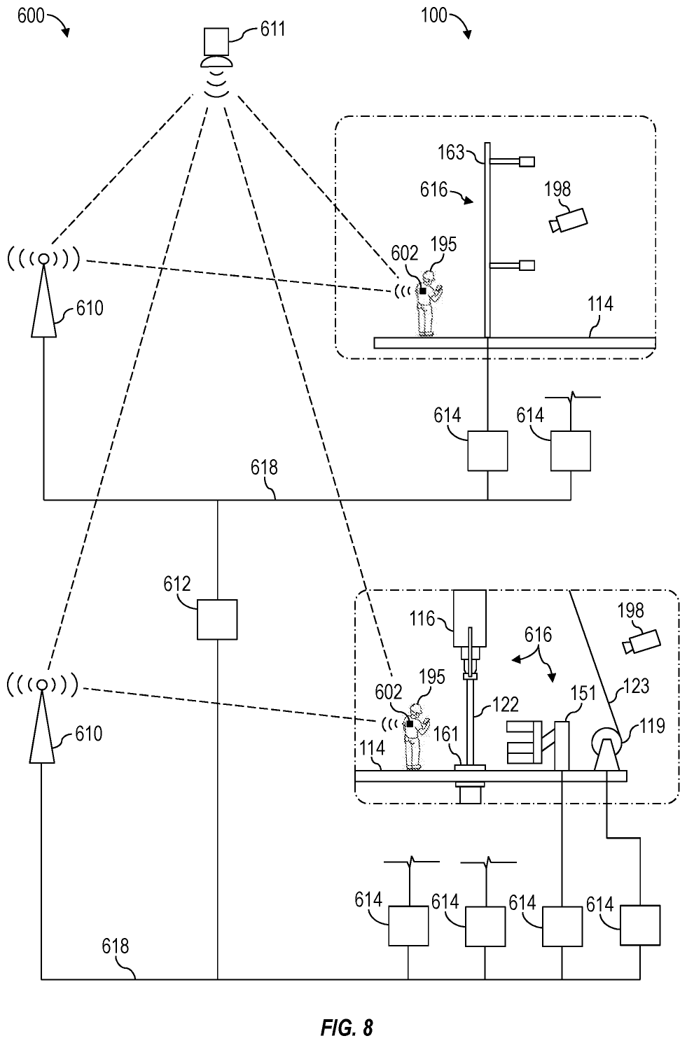

FIG. 8 is a schematic view of example implementation of wearable electronic devices 602 according to one or more aspects of the present disclosure utilized at the well construction system 100 (e.g., drill rig) shown in FIGS. 1 and 2, and communicatively connected with various portions of the well construction system 100 via a wireless communication network 600. The wearable electronic devices 602 may each comprise one or more features and/or modes of operation of the wearable electronic devices 300, 502, 504, 506, 508 shown in FIGS. 3-7. The wearable electronic devices 602 may be disposed in association with wristbands, gloves, safety glasses, safety hats, safety vests, overalls, jackets, and other outerwear to be worn by a human worker 195 (e.g., a roughneck or another wellsite operator). The well construction system 100 represents an example worksite in which the wearable electronic devices 602 according to one or more aspects of the present disclosure may be implemented. Thus, it is to be understood that the wearable electronic devices 602 may be implemented in other well construction systems, mining sites, building construction sites, manufacturing facilities, maintenance (e.g., repair) facilities, and/or other environments in which automated machines or equipment are utilized. The following description refers to FIGS. 1, 2, and 8 collectively.

The wireless communication network 600 may comprise a plurality of wireless access points 610 (e.g., wireless base stations) disposed at various locations of the well construction system 100 and a communication satellite 611 communicatively connected with each other. For example, one or more of the wireless access points 610 may be mounted to the wellsite structure 112, the rig floor 114, and/or other equipment. One or more of the wireless access points 610 may also or instead be mounted at various locations along the wellsite surface 104. A processing device 612 and a plurality of local controllers 614 may be communicatively connected with and operable to control various automated wellsite equipment 616 of the well construction system 100 collectively operable to construct the oil and/or gas wellbore 102. The automated equipment 616 may include, for example, the iron roughneck 151, the PHM 163, the draw works 119 (actuating the vertical movement of the top drive 116), the reciprocating slips 161, the catwalk 131, and the solids and gas control equipment 170, among other examples. The wireless access points 610 may be electrically connected (i.e., wired) with the processing device 612 and the local controllers 614 via a wired communication network 618 and/or via a wireless communication network (not shown). The processing device 612 may be or comprise one or more of the processing devices 192, 202, 320 shown in FIGS. 1-3, respectively, the equipment controllers 614 may be or comprise the local equipment controllers 221-226 shown in FIG. 2, and each wireless access point 610 may be or comprise the transceiver 322 shown in FIG. 3.

Each wearable electronic device 602 may comprise a wireless transmitter and/or receiver (e.g., a transceiver) operable to wirelessly communicate with one or more wireless access points 610 and/or the communication satellite 611. Communication between the wireless access points 610 and the wearable electronic devices 602 may be facilitated via a wireless connection, such as radio frequency signals (e.g., Bluetooth, Wi-Fi, cellular network, and the like). Each wearable electronic device 602 may, thus, be communicatively connected with the processing device 612 and the local controllers 614 via at least the wireless communication network 600 and the wired communication network 618.

The wearable electronic devices 602 and/or the processing device 612 may comprise a processor and a memory device for storing executable computer program code, such as may include machine-readable coded instructions that, when executed by the processor, may cause the wearable electronic devices 602 and/or the processing device 612 to perform or to cause to be performed at least portions of methods and processes described herein. For example, the processor of the wearable electronic devices 602 and/or of the processing device 612 may be operable to receive sensor data generated by sensors of the wearable electronic devices 602, process the sensor data, and generate or otherwise output control commands to output devices of the wearable electronic devices 602 to cause the output devices to output sensory signals to be perceived by the worker 195 based on the sensor data. The processor of the wearable electronic devices 602 and/or of the processing device 612 may also or instead be operable to receive the sensor data generated by the sensors of the wearable electronic devices 602, process the sensor data, and generate or otherwise output control commands to the local controllers 614 to control an associated piece of wellsite equipment 616 based on the sensor data.

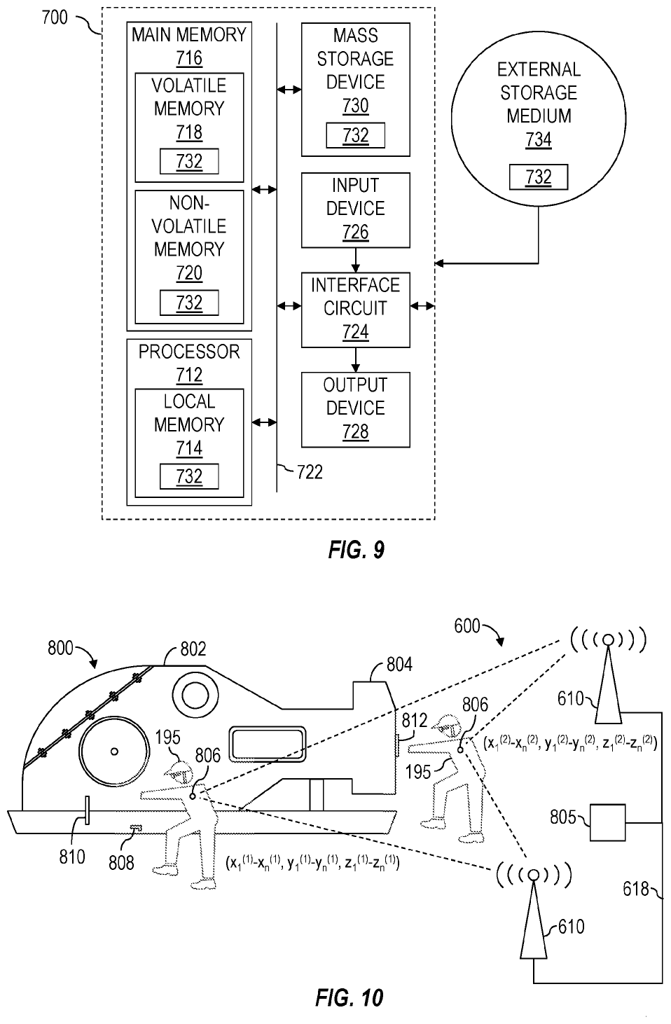

FIG. 9 is a schematic view of at least a portion of an example implementation of a processing device or system 700 according to one or more aspects of the present disclosure. The processing system 700 may be or form at least a portion of one or more of the processing devices 192, 202, 320, 612, the controllers 221-226, 310, 614, and/or the wearable electronic devices 300, 502, 504, 506, 508, 602 shown in one or more of FIGS. 1-8. Accordingly, the following description refers to FIGS. 1-9, collectively.

The processing system 700 may be or comprise, for example, one or more processors, controllers, special-purpose computing devices, PCs (e.g., desktop, laptop, and/or tablet computers), personal digital assistants, smartphones, IPCs, PLCs, servers, internet appliances, and/or other types of computing devices. Although it is possible that the entirety of the processing system 700 is implemented within one device, it is also contemplated that one or more components or functions of the processing system 700 may be implemented across multiple devices, some or an entirety of which may be at the worksite (e.g., wellsite) and/or remote from the worksite.

The processing system 700 may comprise a processor 712, such as a general-purpose programmable processor. The processor 712 may comprise a local memory 714, and may execute machine-readable and executable program code instructions 732 (i.e., computer program code) present in the local memory 714 and/or another memory device. The processor 712 may execute, among other things, the program code instructions 732 and/or other instructions and/or programs to implement the example methods, processes, and/or operations described herein. For example, the program code instructions 732, when executed by the processor 712 of the processing system 700, may cause the processor 712 to receive and process sensor data, and output control commands or other information to one or more portions of the wearable electronic devices to perform example methods and/or operations described herein. The program code instructions 732, when executed by the processor 712 of the processing system 700, may also or instead cause one or more portions or pieces of worksite (e.g., wellsite) equipment to perform the example methods and/or operations described herein. The processor 712 may be, comprise, or be implemented by one or more processors of various types suitable to the local application environment, and may include one or more of general-purpose computers, special-purpose computers, microprocessors, digital signal processors (DSPs), field-programmable gate arrays (FPGAs), application-specific integrated circuits (ASICs), and processors based on a multi-core processor architecture, as non-limiting examples. Examples of the processor 712 include one or more INTEL microprocessors, microcontrollers from the ARM and/or PICO families of microcontrollers, embedded soft/hard processors in one or more FPGAs.

The processor 712 may be in communication with a main memory 716, such as may include a volatile memory 718 and a non-volatile memory 720, perhaps via a bus 722 and/or other communication means. The volatile memory 718 may be, comprise, or be implemented by random access memory (RAM), static random access memory (SRAM), synchronous dynamic random access memory (SDRAM), dynamic random access memory (DRAM), RAMBUS dynamic random access memory (RDRAM), and/or other types of random access memory devices. The non-volatile memory 720 may be, comprise, or be implemented by read-only memory, flash memory, and/or other types of memory devices. One or more memory controllers (not shown) may control access to the volatile memory 718 and/or non-volatile memory 720.

The processing system 700 may also comprise an interface circuit 724, which is in communication with the processor 712, such as via the bus 722. The interface circuit 724 may be, comprise, or be implemented by various types of standard interfaces, such as an Ethernet interface, a universal serial bus (USB), a third generation input/output (3GIO) interface, a wireless interface, a cellular interface, and/or a satellite interface, among others. The interface circuit 724 may comprise a graphics driver card. The interface circuit 724 may comprise a communication device, such as a modem or network interface card to facilitate exchange of data with external computing devices via a network (e.g., Ethernet connection, digital subscriber line (DSL), telephone line, coaxial cable, cellular telephone system, satellite, etc.).

The processing system 700 may be in communication with various video cameras, sensors, actuators, equipment controllers, and other devices via the interface circuit 724. The interface circuit 724 can facilitate communications between the processing system 700 and one or more devices by utilizing one or more communication protocols, such as an Ethernet-based network protocol (such as ProfiNET, OPC, OPC/UA, Modbus TCP/IP, EtherCAT, UDP multicast, Siemens S7 communication, or the like), a proprietary communication protocol, and/or another communication protocol.

One or more input devices 726 may also be connected to the interface circuit 724. The input devices 726 may permit workers 195 to enter the program code instructions 732, which may be or comprise control commands, operational parameters, and/or operational set-points. The program code instructions 732 may further comprise modeling or predictive routines, equations, algorithms, processes, applications, and/or other programs operable to perform example methods and/or operations described herein. The input devices 726 may be, comprise, or be implemented by a keyboard, a mouse, a joystick, a touchscreen, a track-pad, a trackball, an isopoint, and/or a voice recognition system, among other examples. One or more output devices 728 may also be connected to the interface circuit 724. The output devices 728 may permit for visualization or other sensory perception of various data, such as sensor data, status data, and/or other example data. The output devices 728 may be, comprise, or be implemented by video output devices (e.g., an LCD, an LED display, a CRT display, a touchscreen, etc.), printers, and/or speakers, among other examples. The one or more input devices 726 and the one or more output devices 728 connected to the interface circuit 724 may, at least in part, facilitate the HMIs described herein.

The processing system 700 may comprise a mass storage device 730 for storing data and program code instructions 732. The mass storage device 730 may be connected to the processor 712, such as via the bus 722. The mass storage device 730 may be or comprise a tangible, non-transitory storage medium, such as a floppy disk drive, a hard disk drive, a compact disk (CD) drive, and/or digital versatile disk (DVD) drive, among other examples. The processing system 700 may be communicatively connected with an external storage medium 734 via the interface circuit 724. The external storage medium 734 may be or comprise a removable storage medium (e.g., a CD or DVD), such as may be operable to store data and program code instructions 732.

As described above, the program code instructions 732 may be stored in the mass storage device 730, the main memory 716, the local memory 714, and/or the removable storage medium 734. Thus, the processing system 700 may be implemented in accordance with hardware (perhaps implemented in one or more chips including an integrated circuit, such as an ASIC), or may be implemented as software or firmware for execution by the processor 712. In the case of firmware or software, the implementation may be provided as a computer program product including a non-transitory, computer-readable medium or storage structure embodying computer program code instructions 732 (i.e., software or firmware) thereon for execution by the processor 712. The program code instructions 732 may include program instructions or computer program code that, when executed by the processor 712, may perform and/or cause performance of example methods, processes, and/or operations described herein.

The present disclosure is further directed to various methods, processes, and/or operations performed or otherwise facilitated by a wearable electronic device comprising and/or communicatively connected with a processing device executing program code instructions according to one or more aspects of the present disclosure. The wearable electronic device may be utilized, for example, to identify or determine location of a human worker wearing the wearable electronic device. Referring again to FIG. 8, the processing device 612 and/or the processing device of the wearable electronic devices 602 may be operable to utilize trilateration techniques to determine three dimensional (3-D) location of the wearable electronic devices 602 at the wellsite 104, such as by utilizing communication signals transmitted between the wearable electronic devices 602, the wireless access points 610, and/or the communication satellite 611. The 3-D location of the wearable electronic devices 602 can be utilized to determine the location of the workers 195 wearing the wearable electronic devices 602. The 3-D location information may be overlaid onto an engineering 3-D model of the worksite (e.g., drill rig) or a workshop, thereby facilitating determination of the location of the workers 195 with respect to various equipment at the worksite and/or the workshop.

Each piece of equipment at the worksite has a typical set of activities being performed on it either during operations or during maintenance events. A wearable electronic device comprising and/or communicatively connected with a processing device according to one or more aspects of the present disclosure may be further utilized to determine worksite activities being performed by a human worker. For example, the processing device may be operable to build, compile, or otherwise generate a database of activities indicative of operational and/or maintenance events associated with various pieces of equipment at the worksite. Signatures of actions or events performed and/or experienced by the worker in association with various pieces of equipment can be recorded to the database via a wearable electronic device. The recorded signatures may comprise various movements, activities (e.g., type of work or job being performed), events, and/or other attributes including, but not limited to when the activities are performed, time spent for each activity being performed, location of a piece of equipment, orientation of a piece of equipment while the activity is being performed, and movements made by the worker while an activity is being performed, among other examples. The database of signatures may be recorded, compiled, or otherwise generated to identify the type of activity being performed. For example, the signatures may be stored (i.e., recorded) in association with information indicative of the type of worksite activities being performed by the worker. Thereafter, current signatures indicative of physical actions currently performed by the worker that are detected by the sensors of the wearable electronic device may be compared to the stored signatures associated with known worksite activities and/or against a predetermined or planned activity. The current worksite activity performed by the worker may then be determined based on the comparison of the current signatures with the stored signatures and/or the predetermined or planned activity. The database of signatures and other information may be recorded on one or more memory devices, such as the mass storage device 730 and/or the external storage device 734.

FIG. 10 is a schematic side view of example implementation of a mud pump 144 of the well construction system 100 shown in FIG. 1 implemented as a triplex mud pump 800. The following description is directed to generating an example database of activities associated with maintenance events relating to the triplex mud pump 800. The pump 800 is shown comprising a power section 802 and a fluid section 804. The power section 802 is where rotary input motion is converted into reciprocating output, which powers three pistons of the fluid section 804. The fluid section 804 is where the action of the pistons causes drilling fluid to be sucked into fluid chambers and then pressurized to an intended pressure before the drilling fluid is transferred to a discharge manifold.

Each component on the pump 800 has a unique position in 3-D space. Unique signatures of actions or events performed by a human worker 195 on the pump 800 can be recorded on a memory device of a wearable electronic device 806 and/or to a database on a remote memory device 805 (e.g., historian) via the wearable electronic device 806 according to one or more aspects of the present disclosure. The signatures of actions or events performed or experienced by the worker 195 may be communicated to the database via a wireless and/or wired networks 600, 618. The signatures of actions or events performed by the human worker 195 may be compared to signatures of action or events of planned or predetermined maintenance activities, which may also be saved on the wearable electronic device memory and/or the database. The planned or predetermined maintenance activities may comprise, for example, draining and cleaning the power section sump, which includes draining of oil via a crankcase oil drain 808 and refilling the oil through an oil level dipstick port 810, which may be a semi-annual recommendation by the manufacturer. Such activity may include a plurality of movements or actions by the worker 195 (e.g., movements of the wearable electronic device 806) indicated by a series of coordinates (x₁⁽¹⁾-xₙ⁽¹⁾, y₁⁽¹⁾-yₙ⁽¹⁾, z₁⁽¹⁾-zₙ⁽¹⁾) in 3-D space, which will collectively take a certain amount of time on average. The coordinates associated with the activity of maintaining the power section sump may be different from the maintenance activity of removing and cleaning valve covers 812 on the fluid section 804, which may be a bi-weekly recommendation by the manufacturer, indicated by a series of coordinates (x₁⁽²⁾-xₙ⁽²⁾, y₁⁽²⁾-yₙ⁽²⁾, z₁⁽²⁾-zₙ⁽²⁾) in 3-D space. Furthermore, the location and orientation of the worker 195 performing each activity are different. Coordinates of the wearable electronic device 806 in 3-D space may be determined via trilateration techniques, such as by utilizing communication signals transmitted between the wearable electronic devices 806, the wireless access points 610, and/or the communication satellite 611 (shown in FIG. 8). After the database of various signatures (e.g., attributes) at a worksite associated (i.e., linked) with corresponding known activities or other events performed and/or experienced at the worksite (perhaps with other contextual data) is compiled or generated, the movements or actions being currently performed by the worker 195 may be compared to or otherwise analyzed with respect to those stored in the wearable electronic device memory and/or database to determine the activity being currently performed by the worker 195.

Operational benchmarks can be set or otherwise determined for each activity performed at the worksite based on the information compiled in the database. Operational benchmarks can help in operational planning and optimizing maintenance activities. The performed activities captured within the database may be utilized to optimize or otherwise change layout of a maintenance shop or an area on the worksite (e.g., a drill rig) to improve efficiency of the activities that are performed most often.

In response to untimely equipment failures, troubleshooting process may include the following set of steps: 1) identifying symptoms, 2) defining the problem, 3) finding the root cause, 4) selecting the solution and identifying the resources, 5) implementing the solution, and 6) evaluating the success. Different solutions may be attempted, thus, steps 4)-6) may be repeated until the achieved success is as intended or otherwise sufficient. It may be difficult to quantify or predict the amount of time it takes to identify the root cause and implement a solution, thus, making it difficult to predict when a piece of equipment will be available for use. However, the steps of identifying the root cause of a problem and implementing a solution to the problem can be optimized based on past activities performed and recorded in the database when similar symptoms were detected (e.g., seen). Thus, recording the maintenance activities performed in association with or in the context of similar symptoms experienced by the piece of equipment can optimize or improve predictability of the availability of equipment.

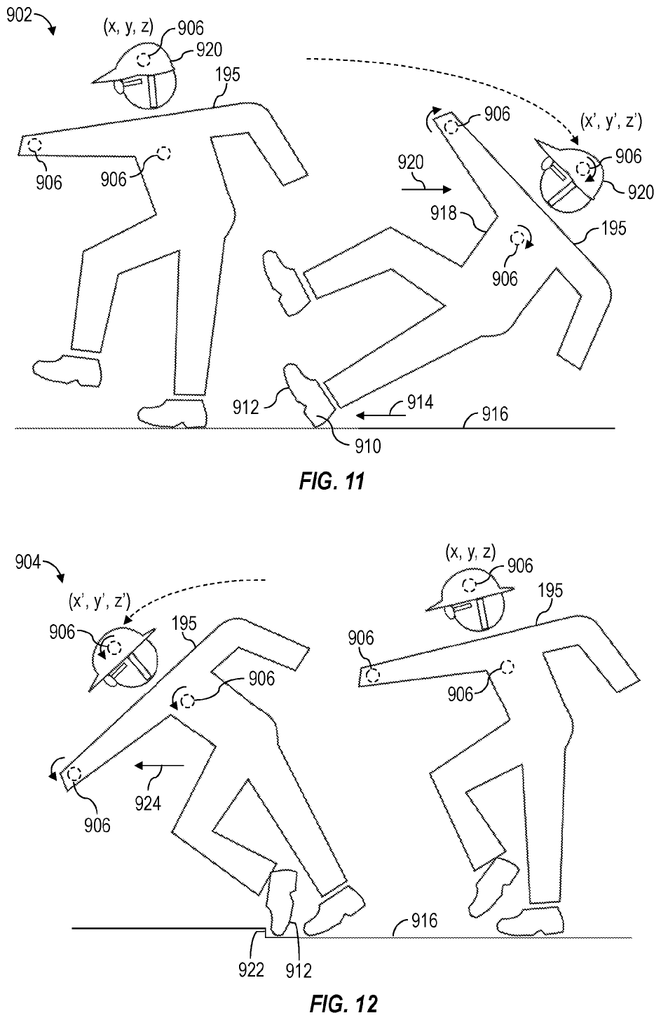

A wearable electronic device comprising and/or communicatively connected with a processing device according to one or more aspects of the present disclosure may be further utilized to detect, identify, and/or determine worksite health, safety, and environment (HSE) incidents, accidents, and/or other events experienced by human workers. FIGS. 11 and 12 are a schematic views of slipping and tripping HSE events 902, 904, respectively, which may be detected, identified, and/or determined by a wearable electronic device and/or a processing device.

As shown in FIG. 11, a slip 902 may be defined as a heel 910 of a foot 912 of a worker 195 moving forward 914 along a floor surface 916 due to inadequate friction while the torso 918 moves backward 920. A slip 902 indicates an undesirable floor condition which may be caused by, for example, a spill or a condition (e.g., black ice or rain) caused by weather. Slipping 902 may result in the worker 195 falling to the floor 916 or the worker 195 may regain their balance. As shown in FIG. 12, a trip 904 may be caused by the foot 912 encountering an obstruction 922 during the swing phase of the foot 912, resulting in the worker's 195 center of mass moving forward 924 beyond the base of support, thereby disrupting the worker's equilibrium. If the balance recovery mechanism does not work, it may result in the worker 195 falling to the floor 916. Both of these events 902, 904 may trigger an HSE investigation. The type, location, and/or time of the HSE events may be determined and/or recorded via one or more wearable electronic devices 906, facilitating corrective actions to be implemented, such as to prevent similar HSE events in the future.

A sensor (e.g., an accelerometer) of a wearable electronic device 906 associated with a safety hat 920 may indicate acceleration and another sensor (e.g., an IMU) of the wearable electronic device 906 associated with the safety hat 920 may indicate direction of movement, which collectively may be utilized to determine nature (i.e., type) of the fall. The same sensors may be implemented in wearable electronic devices 906 associated with a safety vest and/or a wristband, among other examples.

Both magnitude and direction of movement of the wearable electronic devices 906 from an initial position (x, y, z) to a final or otherwise subsequent position (x′, y′, z′) may be tracked to determine both the type and severity of the slip 902 and trip 904. For example, a change in “z” axis may be utilized to determine the magnitude of the movement, and changes in “x” and “y” axes may be utilized to determine the direction and, hence, the type of HSE event. It is to be noted that the rate of change of position and/or rate of change of velocity may also be utilized. For example, change in state of the worker 195 at time t and time t−1 may be determined using one or more of Equations (1)-(3).

$$\begin{matrix}Δ s = ( {x}^{′} - x , {y}^{′} - y , {z}^{′} - z ) & ( 1 ) \\ Δ v = ( \frac{d}{dt} x , \frac{d}{dt} y , \frac{d}{dt} z ) & ( 2 ) \\ Δ a = ( \frac{d}{dt} {v}_{x} , \frac{d}{dt} {v}_{y} , \frac{d}{dt} {v}_{z} ) & ( 3 )\end{matrix}$$

A database of various movements or actions experienced by workers 195 associated (i.e., linked) with information indicative of the type of worksite HSE events (e.g., slips, trips, or other falls) experienced by the workers 195 at the worksite, perhaps with other contextual data, may be compiled or generated. Thereafter, current movements or actions experienced by the worker 195 may be compared to or otherwise analyzed with respect to those stored in the database to determine the worksite HSE event that is currently being experienced by the worker 195. The locations of the recorded worksite HSE events may also be recorded in the database. Remedial measures may be performed at locations associated with frequent worksite HSE events to make those locations safer and, thus, decrease the chances of additional worksite HSE events. Also, the wearable electronic devices 906 may be operated to warn the workers 195 approaching locations of frequent or potential worksite HSE hazards, such as by outputting lights, sounds, vibrations, and/or other sensory signals to be perceived by the workers 195.

When an unintended worksite HSE event, such as a fall or a trip is detected by the wearable electronic device 906, audio and/or video features, such as the video cameras 198 shown in FIGS. 1 and 8, may be automatically activated to record the worksite HSE event. Audio and/or video features (e.g., microphone, video camera) of the wearable electronic device 906 may also or instead be automatically activated to record the worksite HSE event and/or transmit the corresponding sensor signals to a processing device (e.g., processing device 612, 700 shown in FIGS. 9 and 10, respectively). The sensor signals may then trigger an alarm for HSE manager to take appropriate action. The audio and/or video signals may help in the investigation of the circumstances surrounding the worksite HSE event until help arrives. For example, an audio communication established with the affected worker 195 may indicate that he or she is conscious, and no reply may indicate that the worker 195 is unconscious.

Bureau of Labor Statistics published information indicating that around 200,000 cases of workplace back injuries are reported annually, with lower back injuries being the most common form. About two thirds of the lower back injuries were caused by or otherwise associated with manual lifting activities. The primary contributor for such lower back injuries is improper lifting technique. Thus, lower back injuries in the workplace may be reduced by utilizing proper lifting techniques.

A wearable electronic device comprising and/or communicatively connected with a processing device according to one or more aspects of the present disclosure may be further utilized to identify or determine improper lifting techniques at the worksite. When improper lifting technique is utilized by a worker, a wearable electronic device may output a sensory signal (e.g., light, sound, vibration) to warn the wearer of improper lifting technique. The wearable electronic device may be disposed in association with a safety hat, a safety vest, a wristband, and/or other article of clothing.

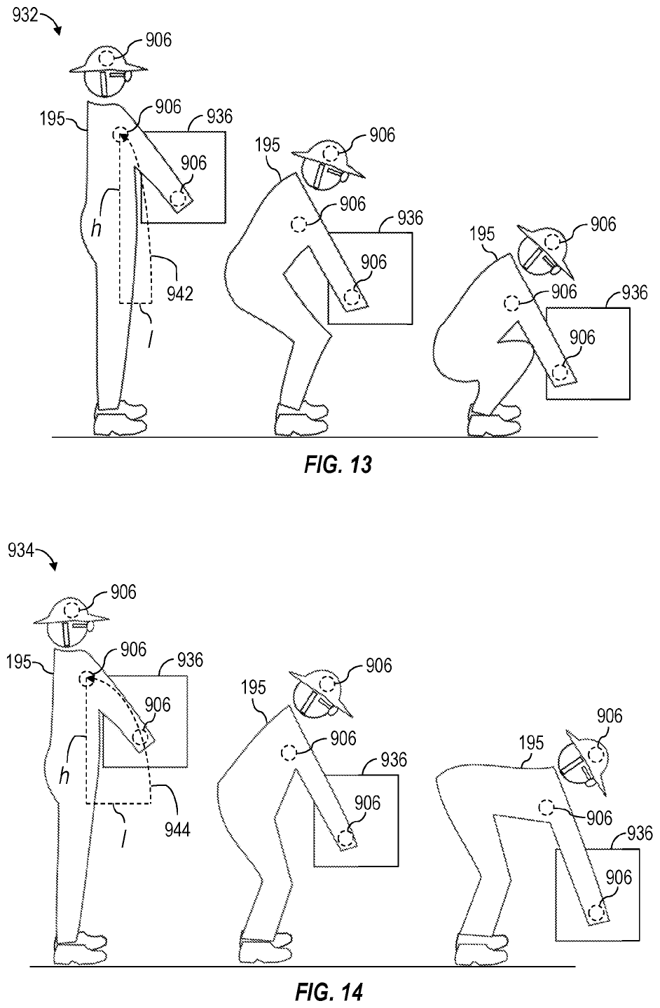

FIGS. 13 and 14 are schematic views of example proper and improper lifting techniques 932, 934, respectively, which may be detected, identified, and/or determined by one or more wearable electronic devices 906 according to one or more aspects of the present disclosure. Each figure shows an initial, an intermediate, and a final position of the proper 932 and improper 934 lifts of an item 936 (e.g., a box, a piece of equipment, a tool, etc.). Each figure further shows a trace of an elliptical trajectory of motion 942, 944 of a wearable electronic device 906 associated with a safety vest or otherwise located in association with a worker's 195 torso while the worker 195 progresses through the positions of each lift 932, 934.

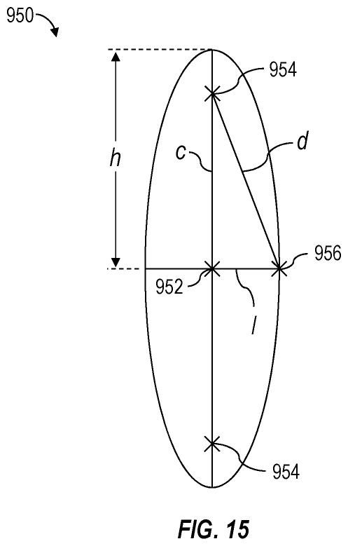

Lifting techniques 932, 934 performed by the worker 195 may be evaluated for proper form by analyzing eccentricity of the elliptical motion 942, 944 of the wearable electronic device 906. FIG. 15 is a schematic view of an ellipse 950 and its components utilized to model the elliptical motion 942, 944 of the wearable electronic device 906 while the worker 195 is lifting the item 936. Symbol h is a vertical distance (i.e., height) the wearable electronic device 906 is moved while the worker 195 is lifting the item 936 and symbol l is the horizontal (i.e., lateral) distance the wearable electronic device 906 is moved while the worker 195 is lifting the item 936. The vertical h and horizontal l distances may be determined by tracking movement of the wearable electronic device 906, such as via an IMU, a GPS receiver, and/or trilateration techniques described above. Symbol c is a distance between the center 952 and a focus 954 of the ellipse 950 and symbol d is a distance between the focus 954 and a vertex 956 of the ellipse 950. Eccentricity e of the ellipse 950 may be determined by utilizing Equation (4).

$$e = \frac{c}{d} \qquad ( 4 )$$

where c²=h²−l². Accordingly, Equation (4) may be rewritten as Equation (5).

$$e = \frac{{( ( h + l ) ( h - l ) )}^{1 \text{/} 2}}{d} \qquad ( 5 )$$

Eccentricity e of the ellipse 950 may be utilized to determine the lateral distance l. Thus, a decrease in eccentricity e results in increase in the horizontal distance l. Accordingly, by computing and monitoring the eccentricity e of lifting motion of a worker 195, proper lifting technique compliance may be monitored and/or enforced.

Published or otherwise known safety statistics or guidelines indicate that for a lifting motion having a vertical distance h of 0.762 meters (2.50 feet), the corresponding horizontal distance l of the lifting motion should be between about 0.03 meters (0.10 feet) and about 0.152 meters (0.50 feet). Utilizing such vertical h and horizontal l distances in Equation (5) indicates that lifting techniques having eccentricity e ranging between about 0.98 and about 1.00 may be deemed as being performed in a safe or otherwise proper manner.

Lifting technique eccentricity e performed by workers 195 at the worksite may be monitored in real-time during a workday. If a processing device determines that a worker 195 is utilizing an improper lifting technique, the processing device may cause the wearable electronic device 906 to output a sensory signal (e.g., light, sound, vibration) to warn the worker 195 of improper lifting technique. Lifting history of each worker 195 may be saved in a database. Frequent history of utilizing improper lifting technique may result in the worker 195 being reprimanded and/or retrained in proper lifting techniques.

A wearable electronic device comprising and/or communicatively connected with a processing device according to one or more aspects of the present disclosure may be further utilized to detect, identify, and/or determine when a worker is in a state of reduced alertness or sleep based on the detected physical actions performed and/or experienced by the worker, and to output sensory signals to induce alertness in the worker. A human head is typically in a state of motion. Such motions are random both in terms of direction and amplitude. During the onset of boredom or sleep, the movement of the head may slow down and then the head tends to move in a particular direction. Head control diminishes while the worker enters a sleeping state. Head and neck muscles relax and the head tends to drop due to gravity. Such movement is controlled by gravity and, thus, rate of change of head position is different than head movements made deliberately by the worker.

Head motions can be recorded by utilizing a wearable electronic device comprising an IMU or another position and/or orientation sensor disposed in association with a safety hat or another wearable article that may be supported by the human head. The sensor may detect and/or record subtle movements of the head along with macro movements, which may be fed into the processing device (e.g., a kinematic analyzer). The processing device may then detect if the worker's head movement is indicative of an alert state or a state of reduced alertness or sleep.

When a wearable electronic device detects a decreasing alertness or loss of alertness, an output device may be caused to output a sensory signal (e.g., light, sound, vibration) to warn the worker that he or she is losing alertness. If the worker does not respond by a way of deliberate movement of the head, other alarms at the worksite may be activated and/or a message may be passed to other worksite personnel to take appropriate action. Such functionality may also be utilized as an “operator presence control” indicator, such as to organize emergency relief in case of a worksite HSE event with respect to the worker wearing the wearable electronic device.

A wearable electronic device comprising and/or communicatively connected with a processing device according to one or more aspects of the present disclosure may be further utilized to change operation of a piece of equipment at the worksite based on determined location of the wearable electronic device at the worksite. With technological advancements in the field of automation, human workers and machines are increasingly working side by side. Traditionally, when machines operate, workers are kept away to minimize the risk of accidents. However, while performing maintenance, workers are in close proximity to the machines. Accordingly, typical safety procedures include locking or shutting down an entire work area or large number of machines to prevent accidents.

A wearable electronic device may facilitate determination of location and/or direction of motion of workers. Accordingly, when a processing device determines that a worker is in close proximity or approaching certain automated machines, the processing device may cause one or more machines to shut down, or change the range, direction, and/or envelope of motion of the machines to accommodate the worker, such as within a predetermined buffer zone around the worker and, thus, permit the worker to perform intended work adjacent the affected machines. The processing device may also or instead generate sensory signals indicative of danger posed by the machines that are near the worker or along the worker's path. The sensory signals outputted by a wearable electronic device may, thus, be based on the determined location of the wearable electronic device.

Wellsite operations often include movement of several pieces of equipment and movement of the drill rig itself. Knowledge of the location and orientation of the wearable electronic device and, hence, the worker wearing it, can help optimize such operations by alerting the worker if he or she is in the critical path defined for the movement of the equipment. The processing device may, thus, be further operable to cause the output devices of a wearable electronic device to generate sensory signals indicative of danger posed by pieces of equipment that are moving toward the worker wearing the wearable electronic device.

In the event of a collision or another accident involving a piece of equipment, the video and/or audio sensors of the wearable electronic device and/or at the worksite, such as the video cameras 198, can be automatically turned on to record the accident. Such trigger can be based on fall detection identified by rapid change in height of the wearable electronic device, which may be detected by sensors (e.g., a piezoelectric sensor, an accelerometer) measuring sudden release of pressure or increased acceleration when the worker falls or the wearable electronic device is dropped. When triggered, the audio and/or video data being streamed to the processing device can be utilized to carry out a timely and appropriate response.

A wearable electronic device comprising and/or communicatively connected with a processing device according to one or more aspects of the present disclosure may be further utilized to direct or instruct movement (e.g., walking, crawling) of the worker wearing the wearable electronic device in a predetermined manner to help the worker reach safety. For example, during major HSE events, such as an explosion, fire, and/or release of poisonous gas, the wearable electronic device may be utilized to help workers escape or mitigate injuries. During a fire in an enclosed space, visibility typically decreases because of smoke and distortion of human senses (e.g., disorientation), which may prevent workers from finding their way out of a building. Similarly, when toxic gasses (e.g., carbon monoxide) fill a room or building, worker senses may be reduced, preventing the workers from finding an exit. In both cases, gases tend to move upward away from the floor. A recommended course of action for avoiding asphyxiation is to crawl along the floor toward an exit. However, under poor or no light conditions, such action may be challenging.

A wearable electronic device comprising a plurality of lights may be utilized during a major HSE event to indicate to a worker in which direction to move. An object map in 3-D may be programmed into a processing device and, perhaps based on the location of work to be accomplished, a subset of such 3-D map may be pushed to the wearable electronic device. Thus, when the worker moves around the worksite (e.g., workspace), the lights on the wearable electronic device may change colors, direction, brightness, etc., thereby indicating, for example, if the worker's face or back is facing an exit and/or if the exit is close by. Thus, even under poor visibility, the lights on the wearable electronic device may help the worker to not just find the exit, but also find the shortest exit point, thereby increasing the chances of survival and decreasing chances of injury.

When a wearable electronic device comprises several sensors and/or other electronic components, electrical power to operate the lights may be reserved unequally, such that the other sensors and/or electronic components do not affect the light functionality. A wearable electronic device may also comprise a beacon, which may facilitate location determination of a worker under adverse condition for a prolonged period of time.

In view of the entirety of the present disclosure, including the figures and the claims, a person having ordinary skill in the art will readily recognize that the present disclosure introduces an apparatus comprising: (A) a wearable electronic device configured to be worn by a human at a worksite, wherein the wearable electronic device comprises: (i) a sensor operable to detect a physical action and/or experience of the human; and (ii) an output device operable to output a sensory signal to be perceived by the human; and (B) a processing device comprising a processor and a memory storing computer program code, wherein the processing device is operable to cause the output device to output the sensory signal based on the detected physical action and/or experience.

The wearable electronic device may comprise the processing device.

The worksite may be a wellsite, a mining site, a building construction site, a manufacturing facility, or a repair facility.

The wearable electronic device may be associated with at least one of a badge, an arm band, a safety hat, safety glasses, a safety vest, overalls, and a jacket.

The sensor may be or comprise a camera, a microphone, an accelerometer, an inertial measurement unit, a locator device, and/or a GPS receiver.

The sensor may be one of a plurality of sensors comprised by the wearable electronic device, and the sensors may be of two or more different types. The different types of sensors may each be selected from the group consisting of a camera, a microphone, an audio speaker, an accelerometer, an inertial measurement unit, a locator device, and a GPS receiver.

The sensory signal may be indicative of another physical action to be performed by the human.

The output device may be or comprise a light emitting device, an audio speaker, and/or a vibration actuator.

The output device may be one of a plurality of output devices comprised by the wearable electronic device, and the output devices may be of two or more different types. The different types of output devices may each be selected from the group consisting of a light emitting device, an audio speaker, and a vibration actuator.

The sensory signal may be visible. For example, the sensory signal may comprise a change in intensity and/or a change in color of a light-emitting device output.

The sensory signal may be audibly and/or tactilely perceptible by the human.

The sensory signal may be or comprise at least one of a light, a sound, and vibrations.

The processing device may be further operable to cause the detected physical action and/or experience to be recorded to a database in association with information indicative of a type of event corresponding to the detected physical action and/or experience.

The processing device may be further operable to cause the detected physical action and/or experience to be recorded to a database in association with information indicative of a type of worksite task being performed by the human and corresponding to the detected physical action and/or experience.

The processing device may be further operable to cause the detected physical action and/or experience to be recorded to a database in association with information indicative of a type of accident corresponding to the detected physical action and/or experience.

The processing device may be further operable to determine a type of event corresponding to the detected physical action and/or experience by comparing the detected physical action and/or experience to previously-recorded physical actions and/or experiences of the human and/or other humans. Each previously-recorded physical action and/or experience may be recorded in association with information indicative of which one of a plurality of types of events corresponds to that previously-recorded physical action and/or experience. The events may include safety incidents and/or equipment maintenance operations.

The processing device may be operable to determine which of previously-planned tasks the human has completed by comparing the detected physical action and/or experience to previously-recorded physical actions and/or experiences associated with the previously-planned tasks. The previously-planned tasks may include maintenance of specific pieces of equipment and/or operating specific pieces of equipment.

The processing device may be further operable to determine what type of worksite task the human is performing based on the detected physical action and/or experience.

The processing device may be operable to determine what type of worksite task the human is performing during the detected physical action and/or experience by comparing the detected physical action and/or experience to previously-recorded physical actions and/or experiences of the human and/or other humans. Each previously-recorded physical action and/or experience may be recorded in association with information indicative of which one of a plurality of types of worksite tasks corresponds to that previously-recorded physical action and/or experience. The types of worksite tasks may include performing maintenance of specific pieces of equipment and/or operating specific pieces of equipment. The processing device may be further operable to determine an amount of time taken to complete the worksite task corresponding to the detected physical action and/or experience.

The processing device may be further operable to determine that the human is performing in an unsafe and/or prohibited manner based on the detected physical action and/or experience. For example, the human may be lifting an object in an unsafe and/or prohibited manner. The processing device may be further operable to cause the output device to output the sensory signal for perception by the human in response to the processing device determining that the human is performing in the unsafe and/or prohibited manner.

The processing device may be further operable to determine that the human is in a state of reduced alertness or sleep based on the detected physical action and/or experience, and the output sensory signal may be for increasing alertness of the human.

The processing device may be further operable to determine that an accident occurred based on the detected physical action and/or experience.

The processing device may be further operable to determine that an accident occurred by comparing the detected physical action and/or experience to previously-recorded physical actions and/or experiences of the human and/or other humans. Each previously-recorded physical action and/or experience may be recorded in association with information indicative of which one of a plurality of types of accidents corresponds to that previously-recorded physical action and/or experience.

The wearable electronic device may further comprise a locator device operable to facilitate determination of location of the wearable electronic device worn by the human at the worksite. An equipment controller at the worksite may be operable to change a mode of operation of a piece of equipment at the worksite based on the determined location of the wearable electronic device at the worksite.

The wearable electronic device may further comprise a locator device operable to facilitate determination of location of the wearable electronic device worn by the human at the worksite. The sensory signal may be further based on the determined location of the wearable electronic device. The sensory signal may be indicative of danger posed by a piece of equipment at the worksite near the determined location of the wearable electronic device.

The processing device may be communicatively connected with a plurality of video cameras at the worksite, and the processing device may be further operable to cause at least one of the video cameras to be operated based on the detected physical action and/or experience.

The processing device may be further operable to cause the output device to output the sensory signal as an indication to the human to move in a predetermined direction during a safety event at the worksite.

The processing device may be further operable to cause the output device to output the sensory signal as an indication to the human to move in a predetermined direction thereby directing the human toward an exit while the human is within an enclosed structure with low visibility.

The processing device may be located at a remote location from the wearable electronic device. The sensor may be operable to generate sensor data indicative of the detected physical action and/or experience. The processing device may be further operable to generate control commands operable to cause the output device to output the sensory signal. The wearable electronic device may further comprise a wireless communicator operable to: transmit the sensor data for reception by the processing device; and receive the control commands generated by the processing device. Communication between the wireless communicator and the processing device may be facilitated by a wireless access point and/or a communication satellite.

The wearable electronic device may comprise a memory device.

The present disclosure also introduces a system comprising: (A) a plurality of wearable electronic devices each configured to be worn by humans at worksites, wherein each wearable electronic device comprises: (i) a sensor operable to detect a human physical action and/or experience; and (ii) an output device operable to output a sensory signal for human perception, wherein the sensory signal is based on at least one of the detected physical actions and/or experiences; (B) a database; and (C) a processing device comprising a processor and a memory storing computer program code, wherein the processing device is communicatively connected with the wearable electronic devices and the database, and wherein the processing device is operable to: (i) record the detected physical actions and/or experiences to the database in association with information indicative of types of worksite events performed and/or experienced by the humans that correspond to the detected physical actions and/or experiences; (ii) compare a subsequent human physical action and/or experience during a corresponding subsequent worksite event, as detected by a sensor of one of the wearable electronic devices, to the recorded physical actions and/or experiences; and (iii) determine the type of the subsequent worksite event based on the comparison.

The worksites may be wellsites, other examples described herein, and/or other sites, locations, or facilities.

Each wearable electronic device may be associated with at least one of a safety hat, safety glasses, a safety vest, overalls, and a jacket.

The sensor of each wearable electronic device may be or comprise at least one of a camera, a microphone, an accelerometer, an inertial measurement unit, a locator device, and a GPS receiver.

Each sensory signal may be indicative of other human physical actions to be performed.

The output device of each wearable electronic device may be or comprise at least one of a light emitting device, an audio speaker, and a vibration actuator.

Each sensory signal may be for human perception via at least one of sight, sound, and touch.

Other aspects of and/or associated with the system may be as described herein.

The present disclosure also introduces a method comprising, while at a wellsite: donning an electronic device that comprises or is in wireless communication with a processing device that includes a processor and a memory storing computer program code; then performing an action or having an experience, wherein the performed action or experience is detected by the donned electronic device; and then perceiving a sensory signal output by the donned electronic device, wherein the sensory signal output is caused by the processing device based on the detected action or experience.

The electronic device may be, be included in, or be attached to at least one of a badge, a safety hat, safety glasses, a safety vest, overalls, and a jacket.

The performed action or experience may be detected via a sensor of the electronic device. The sensor may be or comprise at least one of a camera, a microphone, an accelerometer, an inertial measurement unit, a locator device, and a GPS receiver.

The sensory signal may be indicative of another physical action to be performed by the human, and the method may further comprise performing the other physical action pursuant to the perceived sensory signal.

Perceiving the sensory signal may be via sight, sound, or touch.

Other aspects of and/or associated with the method may be as described herein.

The present disclosure also introduces a method comprising outputting a sensory signal to be perceived by a human wearing an electronic device at a worksite, wherein: the electronic device comprises or is in wireless communication with a processing device that includes a processor and a memory storing computer program code; the electronic device detects physical actions and/or experiences of the human at the wellsite; and the sensory signal output is caused by the processing device based on the detected physical actions and/or experiences.

The worksite may be a wellsite, a mining site, a building construction site, a manufacturing facility, or a repair facility.

The electronic device may be associated with at least one of a safety hat, safety glasses, a safety vest, overalls, and a jacket.

Detecting the physical actions and/or experiences may be performed via a sensor of the electronic device, and the sensor may be or comprise at least one of a camera, a microphone, an accelerometer, an inertial measurement unit, a locator device, and a GPS receiver.

The sensory signal may be indicative of other physical actions to be performed by the human.

Outputting the sensory signal may be performed via an output device of the electronic device. The output device may be or comprise at least one of a light emitting device, an audio speaker, and a vibration actuator.

The sensory signal may be or comprise at least one of a light, a sound, and vibrations.

Other aspects of and/or associated with the method may be as described herein.

The foregoing outlines features of several embodiments so that a person having ordinary skill in the art may better understand the aspects of the present disclosure. A person having ordinary skill in the art should appreciate that they may readily use the present disclosure as a basis for designing or modifying other processes and structures for carrying out the same purposes and/or achieving the same advantages of the embodiments introduced herein. A person having ordinary skill in the art should also realize that such equivalent constructions do not depart from the scope of the present disclosure, and that they may make various changes, substitutions and alterations herein without departing from the spirit and scope of the present disclosure.

The Abstract at the end of this disclosure is provided to permit the reader to quickly ascertain the nature of the technical disclosure. It is submitted with the understanding that it will not be used to interpret or limit the scope or meaning of the claims.

What is claimed is:

1. An apparatus comprising:
a wearable electronic device configured to be worn by a human at a worksite, wherein the wearable electronic device comprises:
a sensor operable to detect a physical action and/or experience of the human; and
an output device operable to output a sensory signal to be perceived by the human; and
a processing device comprising a processor and a memory storing computer program code, wherein the processing device is operable to cause the output device to output the sensory signal based on the detected physical action and/or experience.

2. The apparatus of claim 1 wherein the wearable electronic device is associated with at least one of a badge, an arm band, a safety hat, safety glasses, a safety vest, overalls, and a jacket.

3. The apparatus of claim 1 wherein the sensory signal is indicative of another physical action to be performed by the human.

4. The apparatus of claim 1 wherein the processing device is further operable to cause the detected physical action and/or experience to be recorded to a database in association with information indicative of a type of event corresponding to the detected physical action and/or experience.

5. The apparatus of claim 1 wherein the processing device is further operable to determine a type of event corresponding to the detected physical action and/or experience by comparing the detected physical action and/or experience to previously-recorded physical actions and/or experiences of the human and/or other humans, wherein each previously-recorded physical action and/or experience is recorded in association with information indicative of which one of a plurality of types of events corresponds to that previously-recorded physical action and/or experience.

6. The apparatus of claim 5 wherein the events include safety incidents.

7. The apparatus of claim 5 wherein the events include equipment maintenance operations.

8. The apparatus of claim 1 wherein the processing device is further operable to determine what type of worksite task the human is performing based on the detected physical action and/or experience.

9. The apparatus of claim 1 wherein the processing device is further operable to determine that the human is performing in an unsafe and/or prohibited manner based on the detected physical action and/or experience.

10. The apparatus of claim 9 wherein the processing device is further operable to cause the output device to output the sensory signal for perception by the human in response to the processing device determining that the human is performing in the unsafe and/or prohibited manner.

11. The apparatus of claim 1 wherein the processing device is further operable to determine that the human is in a state of reduced alertness or sleep based on the detected physical action and/or experience, and wherein the output sensory signal is for increasing alertness of the human.

12. The apparatus of claim 1 wherein the processing device is further operable to determine that an accident occurred based on the detected physical action and/or experience.

13. The apparatus of claim 1 wherein the wearable electronic device further comprises a locator device operable to facilitate determination of location of the wearable electronic device worn by the human at the worksite, and wherein an equipment controller at the worksite is operable to change a mode of operation of a piece of equipment at the worksite based on the determined location of the wearable electronic device at the worksite.

14. The apparatus of claim 1 wherein the processing device is further operable to cause the output device to output the sensory signal as an indication to the human to move in a predetermined direction during a safety event at the worksite.

15. A system comprising:
a plurality of wearable electronic devices each configured to be worn by humans at worksites, wherein each wearable electronic device comprises:
a sensor operable to detect a human physical action and/or experience; and
an output device operable to output a sensory signal for human perception, wherein the sensory signal is based on at least one of the detected physical actions and/or experiences;
a database; and
a processing device comprising a processor and a memory storing computer program code, wherein the processing device is communicatively connected with the wearable electronic devices and the database, and wherein the processing device is operable to:
record the detected physical actions and/or experiences to the database in association with information indicative of types of worksite events performed and/or experienced by the humans that correspond to the detected physical actions and/or experiences;
compare a subsequent human physical action and/or experience during a corresponding subsequent worksite event, as detected by a sensor of one of the wearable electronic devices, to the recorded physical actions and/or experiences; and
determine the type of the subsequent worksite event based on the comparison.

16. The system of claim 15 wherein the sensor of each wearable electronic device is or comprises at least one of a camera, a microphone, an accelerometer, an inertial measurement unit, a locator device, and a GPS receiver.

17. The system of claim 15 wherein each sensory signal is indicative of other human physical actions to be performed.

18. The system of claim 15 wherein each sensory signal is for human perception via at least one of sight, sound, and touch.

19. A method comprising:
while at a wellsite:
donning an electronic device that comprises or is in wireless communication with a processing device that includes a processor and a memory storing computer program code;
then performing an action or having an experience, wherein the performed action or experience is detected by the donned electronic device; and
then perceiving a sensory signal output by the donned electronic device, wherein the sensory signal output is caused by the processing device based on the detected action or experience.

20. The method of claim 19 wherein the sensory signal is indicative of another physical action to be performed by the human, and wherein the method further comprises performing the other physical action pursuant to the perceived sensory signal.
